(19) United States
(12) Patent Application Publication
(10) Pub. No.: US 2007/0066615 A1
(43) Pub. Date: Mar. 22, 2007

(54) Heterocyclic compounds

(75) Inventors: Christoph Gerdes, Leverkusen (DE); Elisabeth Perzborn, Wuppertal (DE); Jens Pohlmann, Basel (CH); Susanne Rohrig, Essen (DE); Alexander Straub, Wuppertal (DE); Christian R. Thomas, Wuppertal (DE); Arounarith Tuch, Lyon (FR); Karl-Heinz Schlemmer, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEATHCARE AG, GERMANY (DE)

(21) Appl. No.: 10/557,168

(22) PCT Filed: May 6, 2004
(86) PCT No.: PCT/EP04/04836

§ 371 Date: Oct. 23, 2006

(30) Foreign Application Priority Data
May 19, 2003 (DE) 103-22-469.6

(51) Int. Cl. A61K 31/5377; A61K 31/506; A61K 31/496; A61K 31/501

(52) U.S. Cl. 514/237.5; 514/362; 514/363; 514/364; 514/424; 514/389; 514/376; 514/252.05; 514/256; 514/340; 514/342; 544/122; 544/144; 544/238; 544/333; 546/269.1; 546/268.4; 546/268.7; 546/271.1

(57) ABSTRACT

The present invention relates to the area of blood clotting. The invention relates in particular to certain heterocyclic compounds, to processes for their preparation, to their use for the treatment and/or prophylaxis of diseases, and to their use for producing medicaments for the treatment and/or prophylaxis of diseases.

The present invention relates to the area of blood clotting. The invention relates in particular to certain heterocyclic compounds, to processes for their preparation, to their use for the treatment and/or prophylaxis of diseases, and to their use for producing medicaments for the treatment and/or prophylaxis of diseases.

Blood clotting is a protective mechanism of the body with whose aid defects in the vessel wall can rapidly and reliably be “sealed”. It is thus possible to prevent or minimize a blood loss. The bleeding after injury to a vessel is stopped essentially by the coagulation system in which an enzymatic cascade of complex reactions of plasma protein is induced. Numerous blood clotting factors are involved in this, each of which, as soon as it is activated, converts the respective next inactive precursor into its active form. At the end of the cascade, soluble fibrinogen is converted into insoluble fibrin to result in a blood clot. A distinction is traditionally made in blood clotting between the intrinsic and extrinsic system which coalesce in an final common reaction pathway. Factor Xa, which is formed from the proenzyme factor X, plays a key part in this because it connects the two coagulation pathways. The activated serine protease Xa cleaves prothrombin to thrombin. The resulting thrombin in turn itself cleaves fibrinogen to fibrin. Subsequent crosslinking of the fibrin monomers results in the formation of blood clots and thus stopping the bleeding. In addition, thrombin is a potent inducer of platelet aggregation, which likewise makes a considerable contribution to hemostasis.

Hemostasis is subject to a complex regulatory mechanism. Uncontrolled activation of the coagulation system or a defective inhibition of the activation processes may bring about the formation of local thromboses or embolisms in vessels (arteries, veins, lymphatic vessels) or the cavities of the heart. This may lead to serious thromboembolic disorders. In addition, a hypercoagulability—systemic—in association with a consumptiona coagulopathy may lead to disseminated intravascular coagulation. Thromboembolic complications also occur in association with microangiopathic hemolytic anemias, extracorporeal blood circulations, such as hemodialysis, and heart valve prostheses.

Thromboembolic disorders are the commonest cause of morbidity and mortality in most industrialized countries (Heart Disease; A Textbook of Cardiovascular Medicine, Eugene Braunwald, 5th edition, 1997, W.B. Saunders Company, Philadelphia; Allgemeine und spezielle Pharmakologie und Toxikologie, W. Forth, D. Henschler, W. Rummel, K. Starke, 7th edition, 1996, Spektrum Akademischer Verlag, Heidelberg).

The anticoagulants, i.e. substances for inhibiting or preventing blood clotting, known in the art have various, often serious, disadvantages. An efficient treatment method or prophylaxis of thromboembolic disorders therefore proves in practice to be very difficult and unsatisfactory.

One substance used for the therapy and prophylaxis of thromboembolic disorders is heparin, which is administered parenterally or subcutaneously. Although nowadays low molecular weight heparin is increasingly preferred because of the more favorable pharmacokinetic properties, even with this it is not possible to avoid the known disadvantages which are described below and which exist with heparin therapy. Thus, heparin has no oral activity and has only a comparatively short half-life. Since heparin simultaneously inhibits a plurality of factors in the blood clotting cascade, it has a nonselective effect. In addition, there is a high risk of bleeding, it being possible in particular for cerebral hemorrhages and hemorrhages in the gastrointestinal tract to occur, and thrombopenia, alopecia medicomentosa or osteoporosis is possible (Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”).

A second class of anticoagulants is represented by the vitamin K antagonists. These include for example 1,3-indanediones, but especially compounds such as warfarin, phenprocoumon, dicumarol and other coumarin derivatives which inhibit nonselectively the synthesis of various products of certain vitamin K-dependent coagulation factors in the liver. Owing to the mechanism of action, however, the onset of action is only very slow (latency period of 36 to 48 hours until of the onset of action). Although the compounds can be administered orally, elaborate individual stabilization and observation of the patient is necessary because of the high risks of hemorrhage and the narrow therapeutic index. In addition, further side effects such as gastrointestinal disturbances, hair loss and skin necroses have been described (Pschyrembel, Klinisches Worterbuch, 257th edition, 1994, Walter de Gruyter Verlag, pages 292 et seq., entry “Cumarinderivate”; Ullmann's Encyclopedia of Industrial Chemistry, 5th edition, VCH Verlagsgesellschaft, Weinheim, 1985-1996, entry “Vitamin K”).

Very recently, a new therapeutic approach for the treatment and prophylaxis of thromboembolic disorders has been described. The aim of this new therapeutic approach is to inhibit factor Xa (cf. WO-A-99/37304; WO-A-99/06371; J. Hauptmann, J. Sturzebecher, Thrombosis Research 1999, 93, 203; F. Al-Obeidi, J. A. Ostrem, Factor Xa inhibitors by classical and combinatorial chemistry, DDT 1998, 3, 223; F. Al-Obeidi, J. A. Ostrem, Factor Xa inhibitors, Exp. Opin. Ther. Patents 1999, 9, 931; B. Kaiser, Thrombin and factor Xa inhibitors, Drugs of the Future 1998, 23, 423; A. Uzan, Antithrombotic agents, Emerging Drugs 1998, 3, 189; B.-Y. Zhu, R. M. Scarborbugh, Curr. Opin. Card. Pulm. Ren. Inv. Drugs 1999, 1 (1), 63). In accordance with the central part played by factor Xa in the blood clotting cascade, factor Xa represents one of the principal targets for anticoagulant active ingredients [S. A. V. Raghavan, M. Dikshit, Drugs of the Future 2002, 27, 669-683 “Recent advances in the status and targets of antithrombotic agents”; H. A. Wieland, V. Laux, D. Kozian, M. Lorenz, Current Opinion in Investigational Drugs 2003, 4, 264-271 “Approaches in anticoagulation: Rationales for target positioning”].

It has in this connection been shown that various compounds, both peptides and non-peptides, are effective factor Xa inhibitors in animal models. A large number of direct factor Xa inhibitors is now known [J. M. Walenga, W. P. Jeske, D. Hoppensteadt, J. Fareed, Current Opinion in Investigational Drugs 2003, 4, 272-281 “Factor Xa Inhibitors: Today and beyond”; K. T. Tan, A. Makin, G. Y. H. Lip, Expert Opin. Investig. Drugs 2003, 12, 799-804 “Factor X Inhibitors”; J. Ruef, H. A. Katus, Expert Opin.

Investig. Drugs 2003, 12, 781-797 “New antithrombotic drugs on the horizon”; A. Betz, Recent advances in Factor Xa inhibitors, Expert Opin. Ther. Patents 2001, 11, 1007; M. M. Samama, Synthetic direct and indirect factor Xa inhibitors, Thrombosis Research 2002, 106, 267]. Oxazolidinones having such activity are described for example in WO 01/47919 and WO 02/064575.

It is now an object of the present invention to provide novel substances for controlling disorders having a large therapeutic range.

The present invention relates to compounds of the formula (I) 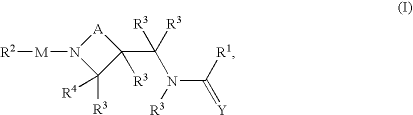
in which
A is a group 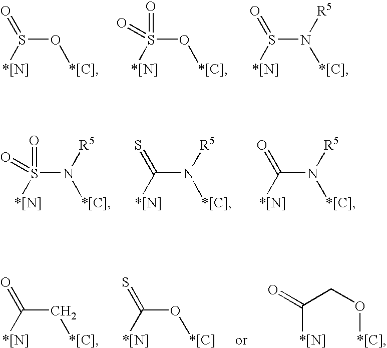
   where
   *[N] is the point of attachment to the nitrogen,
   *[C] is the point of attachment to the carbon, and
   R⁵ is hydrogen or alkyl,
M is an aryl, pyridyl, pyrimidyl, pyridazinyl, thienyl, furyl or pyrrolyl radical which is unsubstituted or is substituted once or twice by radicals selected independently of one another from the group of halogen, trifluoromethyl, trifluoromethoxy, cyano, nitro, carbamoyl, hydroxy, amino, alkylcarbonyl, alkoxycarbonyl, optionally alkylamino-substituted alkylaminocarbonyl, alkylcarbonyloxy, alkyl, alkylamino and alkoxy,
   where
   alkyl, alkylamino and alkoxy in turn may be substituted by amino, hydroxy, alkylamino, alkoxy, heterocyclyl or heterocyclylcarbonyl,
R¹ is an aryl, heteroaryl or heterocyclyl radical which is unsubstituted or is substituted once, twice or three times by radicals selected independently of one another from the group of halogen, optionally amino-substituted alkyl, amino, alkylamino, hydroxy, alkoxy, alkoxycarbonyl, alkylcarbonyl, alkylcarbonyloxy, trifluoromethyl, trifluoromethoxy, trifluoromethylthio, nitro, oxo, carboxyl and cyano,
R2 is an aryl, pyridyl, pyrimidyl or pyridazinyl radical,
   which may be substituted by halogen, amino, alkylamino, alkylsulfonyl or alkylaminosulfonyl,
   or
   is an —N(R⁶)C(O)R⁷, —N(R⁸)C(O)NR⁹R¹⁰, —N(R¹¹)S(O)ₓR¹², 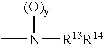
   or —C(O)NR¹⁵R¹⁶ radical,
   where
   R⁶, R⁸, R¹¹, R¹³ and R¹⁵ are independently of one another hydrogen, alkyl or cycloalkyl,
      where
      alkyl and cycloalkyl may in turn be substituted by amino, hydroxy, alkylamino or alkoxy,
   R⁷, R⁹, R¹², R¹⁴ and R¹⁶ are independently of one another alkyl or cycloalkyl,
      where
      alkyl and cycloalkyl may in turn be substituted by amino, hydroxy, alkylamino or alkoxy,
   or
   R⁶ and R⁷ together with the N—C(O) group to which they are bonded form a 4- to 7-membered heterocycle which may also comprise one or two double bonds,
   R⁸ and R⁹ together with the N—C(O)—N(R¹⁰) group to which they are bonded form a 5- to 7-membered heterocycle,
   R¹⁰ is hydrogen, amino, hydroxy, alkylcarbonyl, alkylcarbonyloxy, alkoxycarbonyl, alkylaminocarbonyl, cycloalkyl, alkyl, alkylamino or alkoxy,
      where
      alkyl, alkylamino and alkoxy may in turn be substituted by amino, hydroxy, alkylamino, cycloalkylamino, alkoxy or heterocyclyl,
   R¹¹ and R¹² together with the N—S(O)ₓ group to which they are bonded form a 4- to 7-membered heterocycle which may also comprise one or two double bonds,
   R¹³ and R14 together with the nitrogen to which they are bonded form a 4- to 7-membered heterocycle,
   R¹⁵ and R¹⁶ together with the nitrogen to which they are bonded form a 4- to 7-membered heterocycle,
      where the heterocycle formed by R⁶ and R⁷; R⁸ and R⁹; R¹¹ and R¹²; R¹³ and R¹⁴ or by R¹⁵ and R¹⁶ comprises no, one or two further heteroatoms from the series N, O and/or S and is unsubstituted or is substituted once, twice or three times by radicals independently of one another selected from the group of halogen, trifluoromethyl, cyano, nitro, amino, hydroxy, oxo, alkylcarbonyl, alkylcarbonyloxy, alkoxycarbonyl, alkylamino-carbonyl, alkyl, alkylamino and alkoxy,
         where
         alkyl, alkylamino and alkoxy may in turn be substituted by amino, hydroxy, alkylamino, alkoxy or heterocyclyl,
   x is 1 or 2,
   y is 0 or 1,
R³ is hydrogen or alkyl,
R⁴ is hydrogen, alkoxycarbonyl, alkylaminocarbonyl or alkyl,
   where
   alkyl in turn may be substituted by hydroxy, amino, alkoxy or alkylamino,
Y is O or S
   and the salts, solvates or solvates of the salts thereof.

Compounds of the invention are the compounds of the formula (I) and the salts, solvates and solvates of the salts thereof; the compounds which are encompassed by formula (I) and have the formulae mentioned hereinafter and the salts, solvates and solvates of the salts thereof, and the compounds which are encompassed by formula (I) and are mentioned hereinafter as exemplary embodiments and the salts, solvates and solvates of the salts thereof, where the compounds which are encompassed by formula (I) and are mentioned hereinafter are not already salts, solvates and solvates of the salts.

The compounds of the invention may, depending on their structure, exist in stereo-isomeric forms (enantiomers, diastereomers). The invention therefore relates to the enantiomers or diastereomers and respective mixtures thereof. The stereoisomerically pure constituents can be isolated in a known manner from such mixtures of enantiomers and/or diastereomers.

Where the compounds of the invention may occur in tautomeric forms, the present invention includes all tautomeric forms.

Salts which are preferred for the purposes of the invention are physiologically acceptable salts of the compounds of the invention.

Physiologically acceptable salts of the compounds of the invention include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds of the invention also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and N-methylpiperidine.

Solvates refers for the purposes of the invention to those forms of the compounds which form, in the solid or liquid state, a complex by coordination with solvent molecules. Hydrates are a specific form of solvates in which the coordination takes place with water. Solvates preferred for the purposes of the present invention are hydrates.

For the purposes of the present invention, the substituents have the following meaning, unless specified otherwise: Alkyl per se and “Alk” and “Alkyl” in alkoxy, alkylcarbonyl. alkylamino, alkylamino-carbonyl, alkylaminosulfonyl, alkylsulfonyl, alkoxycarbonyl, alkylcarbonylamino and alkylcarbonyloxy are a linear or branched alkyl radical usually having 1 to 6, preferably 1 to 4, particularly preferably 1 to 3, carbon atoms, by way of example and preferably methyl, ethyl, n-propyl, isopropyl, tert-butyl, n-pentyl and n-hexyl.

Alkoxy is by way of example and preferably methoxy, ethoxy, n-propoxy, isopropoxy, tert-butoxy, n-pentoxy and n-hexoxy.

Alkylcarbonyl is by way of example and preferably acetyl, propanoyl and tert-butanoyl.

Alkylamino is an alkylamino radical having one or two alkyl substituents (chosen independently of one another), by way of example and preferably methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methyl-amino, N-ethyl-N-n-pentylamino and N-n-hexyl-N-methylamino.

Alkylaminocarbonyl is an alkylaminocarbonyl radical having one or two alkyl substituents (chosen independently of one another), by way of example and preferably methylaminocarbonyl, ethylaminocarbonyl, n-propylaminocarbonyl, isopropylaminocarbonyl, tert-butylaminocarbonyl, n-pentylaminocarbonyl, n-hexylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-tert-butyl-N-methylaminocarbonyl, N-ethyl-N-n-pentylaminocarbonyl and N-n-hexyl-N-methylaminocarbonyl.

Alkylaminosulfonyl is an alkylaminosulfonyl radical having one or two alkyl substituents (chosen independently of one another), by way of example and preferably methylaminosulfonyl, ethylaminosulfonyl, n-propylaminosulfonyl, isopropylaminosulfonyl, tert-butylaminosulfonyl, n-pentylaminosulfonyl, n-hexylaminosulfonyl, N,N-dimethylaminosulfonyl, N,N-diethylaminosulfonyl, N-ethyl-N-methylaminosulfonyl, N-methyl-N-n-propylaminosulfonyl, N-isopropyl-N-n-propylaminosulfonyl, N-tert-butyl-N-methylaminosulfonyl, N-ethyl-N-n-pentylaminosulfonyl and N-n-hexyl-N-methylaminosulfonyl.

Alkylsulfonyl is a straight-chain or branched alkylsulfonyl radical. Examples which may be preferably mentioned are: methylsulfonyl, ethylsulfonyl, n-propylsulfonyl, isopropylsulfonyl, tert-butylsulfonyl, n-pentylsulfonyl and n-hexylsulfonyl.

Alkoxycarbonyl is by way of example and preferably methoxycarbonyl, ethoxycarbonyl, n-propoxycarbonyl, isopropoxycarbonyl, tert-butoxycarbonyl, n-pentoxycarbonyl and n-hexoxycarbonyl.

Alkylcarbonyloxy is by way of example and preferably acetoxy and propionyloxy.

Cycloalkyl per se and in cycloalkylamino is a cycloalkyl group usually having 3 to 8, preferably 5 to 7, carbon atoms, by way of example and preferably cyclopropyl, cyclobutyl, cyclopentyl, cyclohexyl and cycloheptyl.

Cycloalkylamino is a cycloalkylamino radical having one or two cycloalkyl substituents (chosen independently of one another), by way of example and preferably cyclopropylamino, cyclobutylamino, cyclopentylamino, cyclohexylamino and cycloheptylamino.

Aryl is a mono-, bi- or tricyclic aromatic, carbocyclic radical usually having 6 to 14 carbon atoms; by way of example and preferably phenyl, naphthyl and phenanthrenyl, in particular phenyl and naphthyl.

Heteroaryl is an aromatic, mono- or bicyclic radical usually having 5 to 10, preferably 5 to 6, ring atoms and up to 4, preferably up to 2, heteroatoms from the series S, O and N, by way of example and preferably thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heterocyclyl per se and heterocyclylcarbonyl is a mono- or polycyclic, preferably mono- or bicyclic, optionally benzo-fused, nonaromatic heterocyclic radical usually having 4 to 7, preferably 5 to 7, ring atoms and up to 3, preferably up to 2, heteroatoms and/or hetero groups from the series N, O, S, SO, SO₂. The heterocyclyl radicals may be saturated or partially unsaturated. 5- to 7-membered, monocyclic saturated heterocyclyl radicals having up to two heteroatoms from the series O, N and S are preferred, such as by way of example and preferably tetrahydrofuranyl, pyrrolidinyl, pyrrolinyl, piperidinyl, piperazinyl, morpholinyl.

Heterocyclylcarbonyl is by way of example and preferably tetrahydrofurancarbonyl, pyrrolidinecarbonyl, pyrrolinecarbonyl, piperidinecarbonyl, piperazinecarbonyl, morpholinecarbonyl.

Halogen is fluorine, chlorine, bromine and iodine.

If radicals in the compounds of the invention are substituted, the radicals may, unless otherwise specified, be substituted one or more times. For the purposes of the present invention, the meaning of all radicals which occur more than once is independent of one another. Substitution by one, two or three identical or different substituents is preferred. Substitution by one substituent is very particularly preferred.

Preference is Given to Compounds of the Formula (I), in which
A is a group 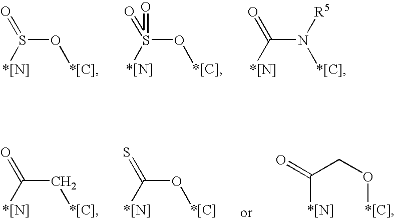
   where
   *[N] is the point of attachment to the nitrogen,
   *[C] is the point of attachment to the carbon, and
   R⁵ is hydrogen or methyl,
M is a phenyl or pyridyl radical which is optionally substituted once by fluorine, chlorine, trifluoromethyl, cyano, nitro, hydroxy, amino, acetyl, alkyl, alkyl-amino or alkoxy,
   where
   alkyl, alkylamino and alkoxy may in turn by substituted by amino, hydroxy, alkylamino, alkoxy or heterocyclyl,
R¹ is a phenyl, pyridyl, thienyl, furyl or pyrrolyl radical which is unsubstituted or is substituted once or twice by radicals selected independently of one another from the group of fluorine, chlorine, bromine, methyl, ethyl, aminomethyl, aminoethyl, amino, alkylamino, hydroxy, methoxy, acetyl, trifluoromethyl, trifluoromethoxy, trifluoromethylthio, nitro and cyano,
R² is a phenyl or pyridyl radical, which may be substituted by fluorine, chlorine, amino or alkylamino,
   or
   is an —N(R⁶)C(O)R⁷, —N(R⁸)C(O)NR⁹R¹⁰, —N(R¹¹)S(O)ₓR¹², 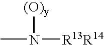
   or —C(O)NR¹⁵ R¹⁶ radical,
   where
   R⁶, R⁷, R⁸, R⁹, R¹¹, R¹², R¹³, R¹⁴ , R¹⁵ and R¹⁶ are independently of one another methyl, ethyl, n-propyl, isopropyl, n-butyl, sec-butyl, isobutyl, tert-butyl, cyclopropyl or cyclopentyl,
      each of which may in turn be substituted by amino, hydroxy, methoxy, ethoxy, methylamino, ethylamino, dimethylamino or diethyl-amino,
   or
   R⁶ and R⁷ together with the N—C(O) group to which they are bonded form a 5-or 6-membered heterocycle which may also comprise one or two double bonds,
   R⁸ and R⁹ together with the N—C(O)—N(R¹⁰) group to which they are bonded form a 5- or 6-membered heterocycle,
   R¹⁰ is hydrogen or alkyl,
      where
      alkyl may in turn be substituted by amino, hydroxy, alkylamino, cycloalkylamino, alkoxy or 5- or 6-membered heterocyclyl,
   R¹¹ and R¹² together with the N—S(O)ₓ group to which they are bonded form a 5- or 6-membered heterocycle which may also comprise one or two double bonds,
   R¹³ and R¹⁴ together with the nitrogen atom to which they are bonded form a 5- or 6-membered heterocycle,
   R¹⁵ and R¹⁶ together with the nitrogen atom to which they are bonded form a 4- to 6-membered heterocycle,
      where the heterocycle formed from R⁶ and R⁷; R⁸ and R⁹; R¹¹ and R^{12;} R¹³ and R¹⁴ or from R¹⁵ and R¹⁶ optionally comprises a further heteroatom from the series N, O and/or S and is unsubstituted or is substituted once or twice by radicals selected independently of one another from the group of amino, hydroxy, oxo, acetyl, alkoxycarbonyl, alkylaminocarbonyl, alkyl, alkylamino and alkoxy,
         where
         alkyl, alkylamino and alkoxy may in turn be substituted by amino, hydroxy, alkylamino, alkoxy or 5- or 6- membered heterocyclyl,
   x is2,
   y is0,
R³ is hydrogen,
R⁴ is hydrogen or alkyl,
   where
   alkyl may in turn be substituted by hydroxy, amino, alkoxy or alkylamino,
y is O,
   and the salts, solvates or solvates of the salts thereof.

Particular preference is given to compounds of the formula (I),

in which

A is a group 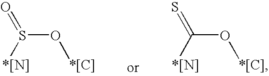
   where
   *[N] is the point of attachment to the nitrogen,
   *[C] is the point of attachment to the carbon,
M is phenyl which is optionally substituted once by fluorine, chlorine, tri-fluoromethyl, cyano, amino, methyl, ethyl, methylamino or dimethylamino,
   where
   methyl and ethyl may in turn be substituted by amino, hydroxy, methylamino, dimethylamino, methoxy, morpholinyl, piperazinyl, piperidinyl or pyrrolidinyl,
R¹ is thienyl which is substituted once by chlorine, bromine or methyl,
R² is a radical 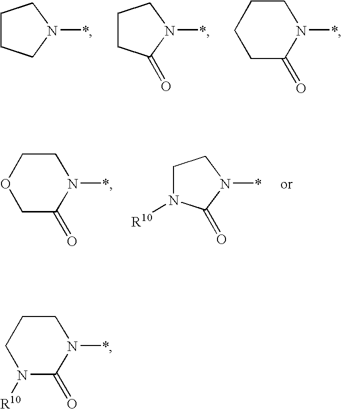
   where
      this radical is unsubstituted or is substituted once or twice by radicals selected independently of one another from the group of amino, hydroxy, methoxy, methylamino and dimethylamino,
   * is the point of attachment to M,
   and
   R¹⁰ is hydrogen, methyl, ethyl or n-propyl,
      where
      ethyl and n-propyl may in turn be substituted by amino, hydroxy, methylamino, ethylamino, cyclopropylamino, isopropylamino, tert-butylamino, dimethylamino, diethylamino, methoxy, ethoxy, morpholinyl, piperazinyl, piperidinyl or pyrrolidinyl,
R³ is hydrogen,
R⁴ is hydrogen,
Y is O,
   and the salts, solvates or solvates of the salts thereof.

The definitions of radicals indicated specifically in the respective combinations or preferred combinations of radicals are replaced irrespective of the particular combinations indicated for the radicals as desired also by definitions of radicals of another combination.

Combinations of two or more of the abovementioned preferred ranges are very particularly preferred.

The invention further relates to a process for preparing the compounds of the invention, which is characterized in that either
[A] compounds of the formula (II) 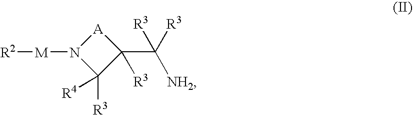
   in which
   A, M, R², R³ and R⁴ have the meanings indicated above,
      are reacted with compounds of the formula (III) 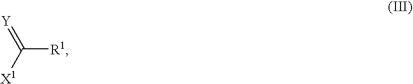
   in which
   Rand Y have the meanings indicated above, and
   X¹is chlorine or hydroxy,
      or
[B] compounds of the formula (IV) 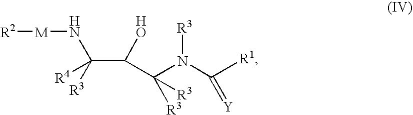
   in which
   M, Rhu 1, R², R³, R⁴ and Y have the meanings indicated above, are reacted
[B1] with compounds of the formula (V) 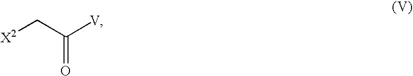
   in which
   V is alkoxy or chlorine, and
   X² is a leaving group, for example chlorine,
      or
[ B2] with thionyl chloride (SOCl₂)
   or
[B3] with thionyl chloride (SOCl₂) and then with an oxidizing agent, for example with sodium periodate,
   or
[B4] with N,N′-thiocarbonyldiimidazole
   or
[C] compounds of the formula (VI) 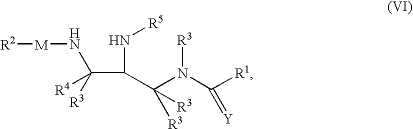
   in which
   M, R¹, R², R³, R⁴, R⁵ and Y have the meanings indicated above, are reacted
[C1] with a carbonic acid equivalent, for example carbonyldiimidazole (CDI),
   or
[C2] with thionyl chloride (SOCl₂)
   or
[C3] with thionyl chloride (SOCl₂) and then with an oxidizing agent, for example sodium periodate,
   or
[C4] with N,N′-thiocarbonyldiimidazole,
   and the resulting compounds of the formula (I) are reacted where appropriate with the appropriate (i) solvents and/or (ii) bases or acids to give the solvates, salts and/or solvates of the salts thereof.

Compounds of the formula (II) can be prepared for example from compounds of the formula of the formula (VII) 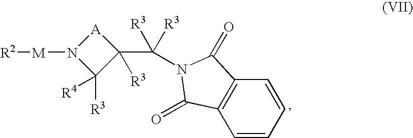
in which
A, M, R², R³ and R⁴ have the meanings indicated above,
   by eliminating the phthalimide protective group.

Compounds of the formula (VII) in turn can be prepared for example
[a] from compounds of the formula (VIII)
   R²—M—NH₂ (VIII),
   in which
   M and R² have the meanings indicated above,
      either
[a1] by reacting with compounds of the formula (IX) 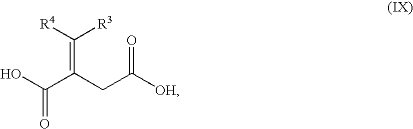
   in which
   R³ and R⁴ have the meanings indicated above,
      to give compounds of the formula (X) 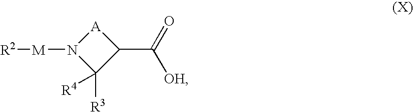
   in which
   A is *[N] —C(O)—CH₂—*[C], and
   *[N], *[C], M, R², R³ and R⁴ have the meanings indicated above,
      subsequent reduction of the carboxyl group to give compounds of the formula (XI) 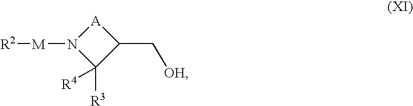
   in which
   A is *[N] —C(O)—CH₂—*[C], and
   *[N], *[C], M, R², R³ and R⁴ have the meanings indicated above,
      and final substitution of the hydroxy group by phthalimide for example under Mitsunobu conditions
      or
[a2] by reacting with compounds of the formula (XII) 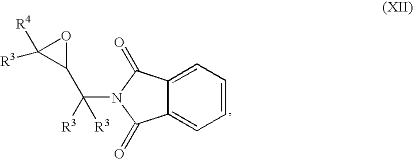
   in which
   R³ and R⁴ have the meanings indicated above,
      to give compounds of the formula (XIII) 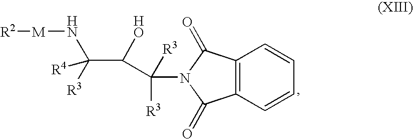
   in which
   M, R², R³ and R⁴ have the meanings indicated above,
      and final reaction with thionyl chloride and, where appropriate, subsequently also with an oxidizing agent
      or
[b] by oxidizing the hydroxy group in compounds of the formula (XIII) to give compounds of the formula (XIV) 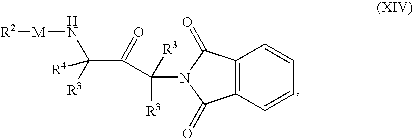
   in which
   M, R², R³ and R⁴ have the meanings indicated above,
      reductive amination of the resulting keto group to give compounds of the formula (XV) 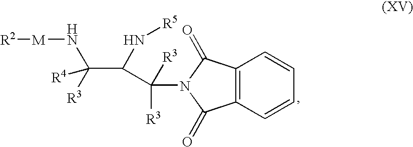
   in which
   M, R² , R³, R⁴ and R⁵ have the meanings indicated above, and final reaction with a carbonic acid equivalent, for example carbonyldiimidazole (CDI), or with thionyl chloride and, where appropriate, subsequently also with an oxidizing agent.

Compounds of the formula (IV) can be prepared for example from compounds of the formula (VIII) by reaction with compounds of the formula (XVI) 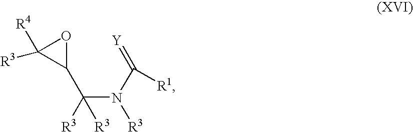
in which
R¹, R³, R⁴ and Y have the meanings indicated above.

Compounds of the formula (VI) can be prepared for example by oxidizing the hydroxy group in compounds of the formula (IV) to give compounds of the formula (XVII) 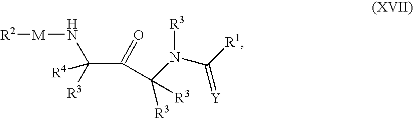
in which
M, R¹, R², R³, R⁴ and Y have the meanings indicated above,
   and subsequent reductive amination of the resulting keto group.

Preparation of the compounds of the invention can be illustrated by the following synthesis scheme.

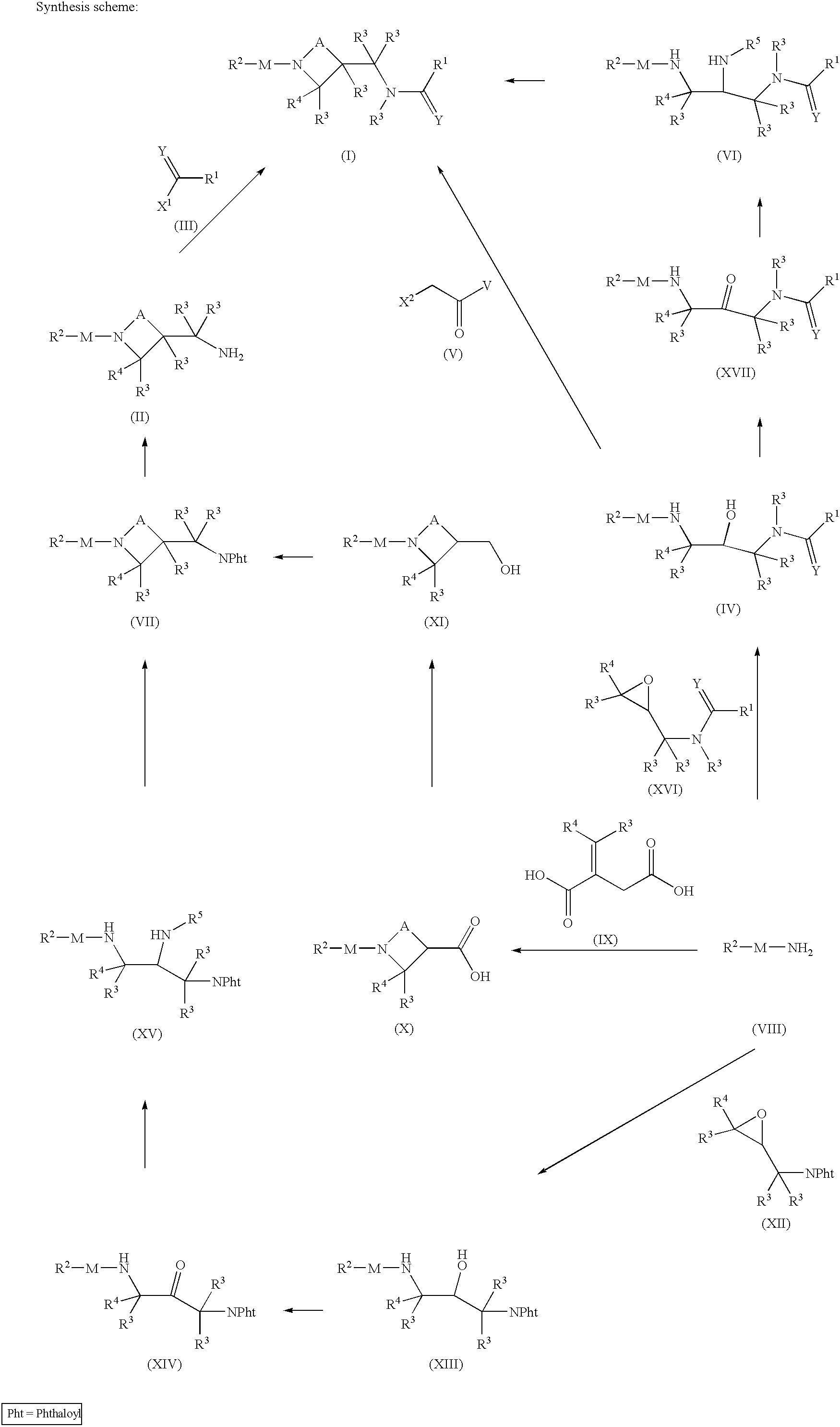

Process step (II)+(III)→(I) preferably takes place in an inert solvent, preferably tetrahydrofuran or dimethylformamide, where appropriate in the presence of auxiliaries and/or bases in a temperature range from 0° C. to the reflux temperature, preferably in the range from 0° C. to room temperature.

Auxiliaries employed for the amide formation are usual condensing agents and/or activating reagents such as carbodiimides, e.g. N′-(3-dimethylaminopropyl)-N-ethylcarbodiimide HCl (EDC), N,N′-dicyclohexylcarbodiimide (DCC), where appropriate in the presence of 1-hydroxy-1H-benzotriazole H₂O (HOBt), benzotriazol-1-yl-oxytrispyrrolidinophosphonium hexafluorophosphate (PyBOP®), 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or carbonyl compounds such as carbonyldiimidazole.

The bases employed are in particular trialkylamines, e.g. triethylamine, N-methylmorpholine (NMM), N-methylpiperidine, N,N-diisopropylethylamine (Hünig's base) or 4-N,N-dimethylaminopyridine (DMAP) or pyridine.

Process step (IV)+(V)→(I) preferably takes place with ethyl chloroacetate or chloroacetyl chloride as (V) in the presence of a base, preferably sodium hydride or potassium tert-butoxide, in an inert solvent, preferably tetrahydrofuran or dimethylformamide at room temperature.

Process steps (IV)+SOCl₂→(I); (VI)+SOCl₂→(I); (XIII)+SOCl₂→(VII); (XV)+SOCl₂→(VII) preferably take place in the presence of N,N-diisopropylethylamine (Hünig's base) as base, in tetrahydrofuran as solvent in a temperature range from −78° C. to room temperature.

Process steps (IV)+SOCl₂+“Ox”→(I); (VI)+SOCl₂+“Ox”→(I); (XIII)+SOCl₂+“Ox”→(VII); (XV)+SOCl₂+“Ox”→(VII) preferably take place in the first step by reaction with thionyl chloride in the presence of N,N-diisopropyl-ethylamine (Hünig's base) as base, in tetrahydrofuran as solvent in a temperature range from −78° C. to room temperature. The subsequent oxidation is preferably carried out with sodium periodate in the presence of ruthenium(III) chloride hydrate in acetonitrile in a temperature range from 0° C. to room temperature.

The cyclization reaction to give cyclic urea derivatives in process steps (VI)→(I) and (XV)→(VII) preferably take place with carbonyldiimidazole (CDI) as carbonic acid equivalent in the presence of 4-N,N-dimethylaminopyridine (DMAP) as base in tetrahydrofuran as solvent in a temperature range from room temperature to 80° C.

The cyclization reaction to give oxazolidinethiones in process step (IV)→(I) and to give imidazolidinethiones in process step (VI)→(I) preferably takes place with N,N′-thiocarbonyldiimidazole in the presence of 4-N,N-dimethylaminopyridine (DMAP) as base in dimethylformamide or tetrahydrofuran as solvent in a temperature range from room temperature to 80° C.

Elimination of the phthalimide protective group in process step (VII)→(II) preferably takes place with hydrazine hydrate or methylamine in methanol or ethanol as solvent in a temperature range from room temperature to 80° C.

Process step (VIII)+(IX)→(X) preferably takes place in aqueous solution under reflux.

Reaction of the carboxyl group to give the corresponding alcohol in process step (X)→(XI) preferably takes place via the stage of the corresponding methyl ester by reacting (X) with thionyl chloride in methanol at 0° C. and subsequent reduction of the resulting methyl ester with sodium borohydride in methanol under reflux to give (XI).

Process step (XI)→(VII) (Mitsunobu reaction) preferably takes place by reacting (XI) with phthalimide in the presence of triphenylphosphine and azodicarboxylates such as, for example, diethyl azodicarboxylate (DEAD) in tetrahydrofuran in a temperature range from 0° C. to room temperature.

Process steps (VIII)+(XII)→(XIII) and (VIII)+(XVI)→(IV) preferably take place with primary amine or aniline derivatives in 1,4-dioxane, 1,4-dioxane/water mixtures, ethanol or ethanol/water mixtures in a temperature range from room temperature to 80° C. or alternatively in the presence of catalytic amounts of ytterbium(III) trifluoromethanesulfonate in tetrahydrofuran in a temperature range from room temperature to 80° C.

Oxidation of the alcohol function to the corresponding ketone in process steps (XIII)→(XIV) and (IV)→(XVII) preferably takes place under the conditions of the Swern oxidation with dimethyl sufoxide and oxalyl chloride or analogous methods based on activated DMSO, such as, for example, with dimethyl sulfoxide and trifluoroacetic anhydride or dimethyl sufoxide and N,N′-dicyclohexylcarbodiimide/-phosphoric acid (Pfitzner-Moffat oxidation).

Reductive amination of the keto function in process steps (XIV)→(XV) and (XVII)→(VI) preferably takes place with sodium cyanoborohydride as reducing agent in the presence of acetic acid and molecular sieves (4Å) in methanol.

Compounds of the formula (III), (V), (VIII), (IX), (XII) and (XVI) are known to the skilled worker per se or can be prepared by customary processes known from the literature.

The compounds of the invention show a valuable range of pharmacological effects which could not have been predicted.

They are therefore suitable for use as medicaments for the treatment and/or prophylaxis of diseases in humans and animals.

The compounds of the invention are selective inhibitors of blood clotting factor Xa which act in particular as anticoagulants.

The present invention further relates to the use of the compounds of the invention for the treatment and/or prophylaxis of disorders, preferably of thromboembolic disorders and/or thromboembolic complications.

The “thromboembolic disorders” within the meaning of the present invention include in particular disorders such as myocardial infarction with ST segment elevation (STEMI) and without ST segment elevation (non-STEMI), stable angina pectoris, unstable angina pectoris, reocclusions and restenoses after coronary interventions such as angioplasty or aortocoronary bypass, thrombotic and thromboembolic stroke, transient ischemic attacks, peripheral arterial occlusive diseases, pulmonary embolisms, deep vein thromboses and renal vein thromboses.

The compounds of the invention are additionally suitable for the treatment of disseminated intravascular coagulation (DIC).

Thromboembolic complications also occur in association with microangiopathic hemolytic anemias, extracorporeal blood circulations, such as hemodialysis, and heart valve prostheses.

The compounds of the invention are additionally also suitable for the prophylaxis and/or treatment of atherosclerotic vascular disorders and inflammatory disorders such as rheumatic disorders of the locomotor system, and additionally likewise for the prophylaxis and/or treatment of Alzheimer's disease and neoplastic disorders such as cancer.

The compounds of the invention may additionally also be employed for preventing coagulation ex vivo, e.g. in stored blood or biological samples containing factor Xa.

The present invention further relates to the use of the compounds of the invention for the treatment and/or prophylaxis of disorders, especially of the aforementioned disorders.

The present invention further relates to the use of the compounds of the invention for producing a medicament for the treatment and/or prophylaxis of disorders, especially of the aforementioned disorders.

The present invention further relates to a method for the treatment and/or prophylaxis of disorders, especially of the aforementioned disorders, by using an amount which has anticoagulant activity of the compound of the invention.

The present invention further relates to a method for preventing blood coagulation in vitro, especially in stored blood or biological samples containing factor Xa, which is characterized in that an amount having anticoagulant activity of the compound of the invention is added.

The present invention further relates to medicaments comprising a compound of the invention and one or more further active ingredients, in particular for the treatment and/or prophylaxis of the aforementioned disorders. Suitable combination active ingredients which may be mentioned by way of example and preferably are:
lipid-lowering agents, especially HMG-CoA (3-hydroxy-3-methylglutaryl-coenzym A) reductase inhibitors,
coronary therapeutics/vasodilators, especially ACE (angiotensin converting enzyme) inhibitors; All (angiotensin II) receptor antagonists; β-adrenoceptor-antagonists; alpha-1-adrenoceptor antagonists; diuretics; calcium channel blockers; substances which bring about an increase in cyclic guanosine monophosphate (cGMP), such as, for example, stimulators of soluble guanylate cyclase;

Page 35; line 14:
plasminogen activators (thrombolytics/fibrinolytics) and compounds which increase thrombolysis/fibrinolysis, such as inhibitors of plasminogen activator inhibitor (PAI inhibitors) or inhibitors of thrombin-activated fibrinolysis inhibitor (TAFI);
substances having anticoagulant activity (anticoagulants);
platelet aggregation-inhibiting substances (platelet aggregation inhibitors);
fibrinogen receptor antagonists (glycoprotein IIb/IIIa antagonists).

The present invention further relates to medicaments which comprise a compound of the invention, normally together with one or more pharmaceutically acceptable excipients, and to the use thereof for the aforementioned purposes.

The compound of the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or otic route or as implant or stent.

The compound of the invention can be administered in suitable administration forms for these administration routes.

Administration forms suitable for oral administration are those which function according to the state of the art and deliver the compound of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilisates, capsules, sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous or intraperitoneal). Administration forms suitable for parenteral administration are, inter alia, injection and infusion preparations in the form of solutions, suspensions, emulsions, lyophilisates or sterile powders.

Examples suitable for other administration routes are medicinal forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, milk, pastes, foams, dusting powders, implants or stents.

The compound of the invention can be converted in a manner known per se into the stated administration forms. This can take place by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. antioxidants such as, for example, ascorbic acid), colors (e.g. inorganic pigments such as, for example, iron oxides) and masking tastes and/or odors.

It has generally proved advantageous on parenteral administration to administer amounts of about 0.001 to 10 mg/kg, preferably about 0.1 to 1 mg/kg, of body weight per day to achieve effective results. The amount per day on oral administration is about 0.01 to 50 mg/kg, preferably 0.1 to 4 mg/kg, of body weight.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The percentage data in the following tests and examples are, unless indicated otherwise, percentages by weight; parts are parts by weight. Solvent ratios, dilution ratios and concentration data for liquid/liquid solutions are in each case based on volume.

### A. EXAMPLES

Abbreviations and Acronyms:

decomp. decomposition
DCI direct chemical ionization (in MS)
DMF N,N-dimethylformamide
DMSO dimethyl sulfoxide
eq equivalent(s)
ESI electrospray ionization (in MS)
h hour(s)
HPLC high pressure, high performance liquid chromatography
LC-MS coupled liquid chromatography-mass spectroscopy
m.p. melting point
MS mass spectroscopy
NMR nuclear magnetic resonance spectroscopy
R_{f} retention index (in TLC)
RP reverse phase (in HPLC)
RT room temperature
Rₜ retention time (in HPLC)
THF Tetrahydrofuran
TLC thin layer chromatography
   LC-MS and HPLC Methods:
   Method 1:

MS instrument type: Micromass ZQ; HPLC instrument type: Waters Alliance 2795; column: Merck Chromolith SpeedROD RP-18e 50 mm×4.6 mm; eluent A: water+500 μl of 50% formic acid per 1 of water; eluent B: acetonitrile+500 μl of 50% formic acid per 1 of acetonitrile; gradient: 0.0 min 10% B→3.0 min 95% B→4.0 min 95% B; oven: 35° C; flow rate: 0.0 min1.0 ml/min→3.0 min 3.0 ml/min→4.0 min 3.0 ml/min; UV detection: 210 nm.

Method 2:

MS instrument type: Micromass ZQ; HPLC instrument type: HP 1100 series; UV DAD; column: Grom-Sil 120 ODS-4 HE 50 mm×2 mm, 3.0 μm; eluent A: water+500 μl of 50% formic acid per 1 of water, eluent B: acetonitrile+500 μl of 50% formic acid per 1 of acetonitrile; gradient: 0.0 min 0% B→2.9 min 70% B→3.1 min 90% B→4.5 min 90% B; oven: 50° C; flow rate: 0.8 ml/min; UV detection: 210 μnm.

Method 3:

Column: Symmetry C18, 2.1 mm×150 mm; eluent A: acetonitrile, eluent B: 0.6 g of 30% HCl per 1of water; gradient: 0.0 min 10% A→4.0 min 90% A→9.0 min 90% A; oven: 50° C; flow rate: 0.6 ml/min; UV detection: 210 nm.

Method 4:

MS instrument type: Micromass ZQ; HPLC instrument type: Waters Alliance 2795; column: Phenomenex Synergi 2μHydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A, flow rate 1 ml/min→2.5 min 30% A, flow rate 2 ml/min→3.0 min 5% A, flow rate 2 ml/min→4.5 min 5% A, flow rate 2 ml/min; oven: 50° C; UV detection: 210 nm.

Method 5:

MS instrument type: Micromass ZQ; HPLC instrument type: HP 1100 series; UV DAD; column: Phenomenex Synergi 2μ Hydro-RP Mercury 20 mm×4 mm; eluent A: 1 l water+0.5 ml of 50% formic acid, eluent B: 1 l of acetonitrile+0.5 ml of 50% formic acid; gradient: 0.0 min 90% A, flow rate 1 ml/min →2.5 min 30% A, flow rate 2 ml/min→3.0 min 5% A, flow rate 2 ml/min→4.5 min 5% A, flow rate 2 ml/min; oven: 50° C; UV detection: 210 nm.

Method 6:

Instrument: Micromass Platform LCZ with HPLC Agilent series 1100; column: Phenomenex Synergi 2μ Hydro-RP Mercury 20 mm×4 mm; Eluent A: 1 l water+0.5 ml of 50% formic acid, eluent B: 1l of acetonitrile+0.5 ml of 50% formic acid; gradient: 0.0 min 90% A, flow rate 1 ml/min→2.5 min 30% A, flow rate 2 ml/min →3.0 min 5% A, flow rate 2 ml/min→4.5 min 5% A, flow rate 2 ml/min; oven: 50° C; UV detection: 210 nm.

Method 7:

Instrument: Micromass Quattro LCZ, with HPLC Agilent series 1100; column: Grom-Sil 120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C; flow rate: 0.8 ml/min; UV detection: 208-400 nm.

Method 8:

Instrument: Micromass Quattro LCZ, HP1100; column: Symmetry C18, 50 mm×2.1 mm, 3.5 μm; eluent A: acetonitrile+0.1% formic acid, eluent B: water+0.1% formic acid; gradient: 0.0 min 10% A→4.0 min 90% A→6.0 min 90% A; oven: 40° C; flow rate: 0.5 ml/min; UV detection: 208-400 nm.

Starting Compounds:

### Example 1A

### 5-Chlorothiophene-2-carbonyl chloride

Obtainable by reacting 5-chlorothiophene-2-carboxylic acid with thionyl chloride, see R. Aitken et aL, Arch. Pharm. (Weinheim Ger.), 1998, 331, 405-411.

### Example 2A

### 1 -(4-Aminophenyl)pyrrolidin-2-one

Obtainable by reducing 1-(4-nitrophenyl)-2-pyrrolidinone, see Reppe et al., Justus Liebigs Ann. Chem. 1955, 596, 209.

### Example 3A

### 4-(4-Aminophenyl)morpholin-3-one

Obtainable by substituting 4-fluoronitrobenzene with morpholin-3-one (J.-M. Lehn, F. Montavon, Helv. Chim. Acta 1976, 59, 1566-1583) and subsequently reducing the 4-(4-morpholin-3-onyl)nitrobenzene (see WO 01/47919, starting compounds I and II, pages 55-57).

### Example 4A

### 1 -(4-Aminophenyl)imidazolidin-2-one

2.0 g (9.6 mmol) of 1-(4-nitrophenyl)imidazolidin-2-one [obtainable by Mitsunobu reaction of 1-(2-hydroxyethyl)-3-(4-nitrophenyl)urea, see T. H. Kim, G. J. Lee, M.-H Cha, Synth. Commun. 1999, 29, 2753-2758] are dissolved in 20 ml of DMF/THF (1:1), mixed with 200 mg of palladium on activated carbon (5%) and hydrogenated. After 12 hours, the reaction mixture is filtered with Tonsil through Celite with suction, washed with THF, concentrated and dried under high vacuum.

Yield: 1.7 g (93% of theory)

LC-MS (method 7): Rₜ=0.31 min.

MS (ESIpos): m/z =178[M+H]⁺.

### Example 5A

### 1-(4-Aminophenyl)-3-(2{[tert-butyl(diphenyl)silyl]oxy}ethyl)tetrahydro-2(1H)-pyrimidinone

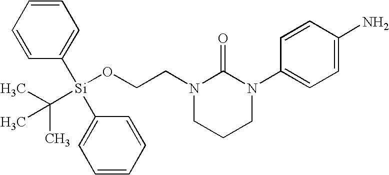

### Stage a): 1-(2-{[tert.-Butyl(diphenyl)silyl]oxy}ethyl)tetrahydro-2(1H)-pyrimidinone

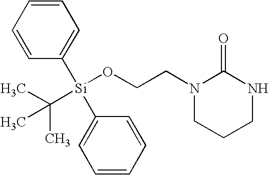

10 g (69.4 mmol) of 1-(2-hydroxyethyl)tetrahydro-2(1H)-pyrimidinone are dissolved in 300 ml of DMF and, at RT, 14.4 ml (104 mmol) of triethylamine, 423.7 mg (3.5 mmol) of 4-N,N-dimethylaminopyridine and 21.1 ml (90.2 mmol) of tert-butylchlorodiphenylsilane are added. The solution is stirred at RT for 24 hours. The residue after concentration of the solution is mixed with water and extracted with dichloromethane. The organic solution is dried and concentrated. Chromatography on silica gel (mobile phase: ethyl acetate, then methanol) results in 24.2 g (91% of theory) of the desired product.

LC-MS (method 1): Rt=2.68 min.

MS (ESIpos): m/z=383 [M+H]⁺

¹H-NMR (200 MHz, CDCl₃): δ=7.70-7.62 (m, 4H), 7.48-7.32 (m, 6H), 4.75 (br, 1H), 3.82 (t, 2H), 3.52-3.37 (m, 4H), 3.32-3.22 (m, 2H), 1.96-1.83 (m, 2H), 1.05 (s, 9H).

### Stage b): 1-(2-{[tert-Butyl(diphenyl)silyl]oxy}ethyl)-3-(4-nitrophenyl)-tetrahydro-2(1H)-pyrimidinone

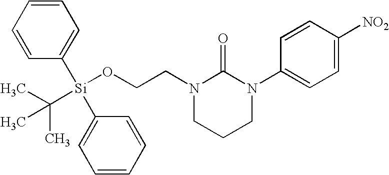

5 g (13 mmol) of 1-(2-{[tert-butyl(diphenyl)silyl]oxy}ethyl)tetrahydro-2(1H)-pyrimidinone are dissolved in 60 ml of DMF in an ultrasonic bath and, at RT under argon, 2.18 g (19.4 mmol) of potassium tert-butoxide are added. After 45 minutes, 2.21 g (15.5 mmol) of 1-fluoro-4-nitrobenzene are added in portions. The solution is stirred at RT overnight, and then ethyl acetate and sodium bicarbonate solution are added. After the extraction, the organic phase is washed with saturated sodium chloride solution, dried and concentrated. Chromatography on silica gel (mobile phase: cyclohexane/ethyl acetate 20:1 and 3:1) results in 2.44 g (37% of theory) of the desired product.

LC-MS (method 1): Rₜ=3.10 min.

MS (ESIpos): m/z=504 [M+H]⁺

¹H-NMR (300 MHz, DMSO-d₆): δ=8.17 (dd, 2H), 7.69-7.63 (m, 4H), 7.49-7.33 (m, 8H), 3.91 (t, 2H), 3.74 (t, 2H), 3.58 (t, 2H), 3.55 (t, 3H), 2.17-2.07 (m, 2H), 1.06 (s, 9H).

### Stage c): 1-(4-Aminophenyl)-3-(2-{[tert-butyl(diphenyl)silyl]oxy}ethyl)-tetrahydro-2(1H)-pyrimidinone

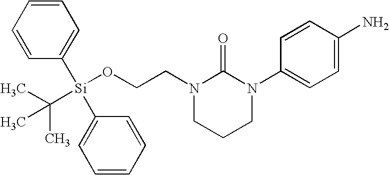

7.80 g (15.5 mmol) of 1-(2-{[tert-butyl(diphenyl)silyl]oxy}ethyl)-3-(4-nitrophenyl)-tetrahydro-2(1H)-pyrimidinone are dissolved in THF and mixed with 2.0 g of palladium on activated carbon (5%) and hydrogenated. After 6 hours, the reaction mixture is filtered with Tonsil through Celite with suction, washed with THF, concentrated and dried under high vacuum.

Yield: 7.34 g (100% of theory)

LC-MS (method 1): Rₜ=2.56 min.

MS (ESIpos): nl/z=474 [M+H]⁺

¹H-NMR (300 MHz, CDCl₃): δ=7.70-7.64 (m, 4H), 7.44-7.34 (m, 6H), 7.06-7.00 (m, 2H), 6.65-6.61 (m, 2H), 3.78 (t, 2H), 3.62-3.50 (m, 6H), 2.10-2.00 (m, 2H), 1.43 (s, 9H).

### Example 6A

### 5-Chloro-N-[(2S)-2-oxiranylmethyl]-2-thiophenecarboxamide

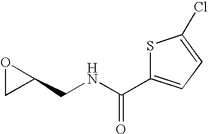

### Stage a): 5-Chloro-N-((S)-2,3-dihydroxypropyl)thiophene-2-carboxamide

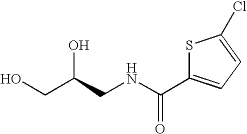

461 g of sodium bicarbonate and 350 g of (2S)-3-aminopropane-1,2-diol hydrochloride are introduced into 2.1 l of water at 13-15° C., and 950 ml of 2-methyl-tetrahydrofuran are added. 535.3 g of 5-chlorothiophene-2-carbonyl chloride (about 93% pure) in 180 ml of toluene are added dropwise over a period of 2 hours to the mixture while cooling at 15-18° C. For workup, the phases are separated, and a total of 1.5 l of toluene is added in several portions to the organic phase. The precipitated product is filtered off with suction, washed with ethyl acetate and dried.

Yield: 593.8 g (91.8% of theory)

m.p.: 114-114.5° C.

### Stage b): N-[(2S)-3-Brom-2-hydroxypropyl]-5-chloro-2-thiophenecarboxamide

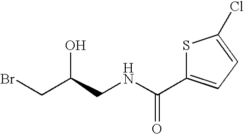

301.7 ml of 33% strength solution of hydrobromic acid in acetic acid are added over a period of 30 minutes to a suspension of 100 g of 5-chloro-N-((S)-2,3-dihydroxy-propyl)-thiophene-2-carboxamide in 250 ml of glacial acetic acid at 21-26° C. Then 40 ml of acetic anhydride are added, and the reaction mixture is stirred at 60-65° C. for 3 hours. Then, at 20-25° C., 960 ml of methanol are added over a period of 30 minutes. The reaction mixture is stirred under reflux for 2.5 hours and then at 20-25° C. overnight. For workup, the solvents are distilled off in vacuo under about 95 mbar. 50 ml of 1-butanol and 350 ml of water are added to the remaining suspension. The precipitated product is filtered off with suction, washed with water and dried.

Yield: 89.8 g (70.9% of theory)

m.p.: 120° C.

### Stage c): 5-Chloro-N-[(2S)-2-oxiranylmethyl]-2-thiophenecarboxamide

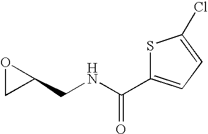

Powdered potassium carbonate (30.8 g, 138.2 mmol) is added to a solution of N-[(2S)-3-bromo-2-hydroxypropyl]-5-chloro-2-thiophenecarboxamide (9.51 g, 31.9 mmol) in dichloromethane (510 ml) at RT, and the reaction mixture is stirred for three days. It is then filtered through a filter layer, the filter layer is washed with dichloromethane, and the filtrate is concentrated in vacuo at RT.

Yield: 7 g (93% of theory)

LC-MS (method 2): Rₜ=2.57 min.

MS (ESIpos): m/z=218 [M+H]⁺

¹H-NMR (300 MHz, DMSO-d₆): δ=8.78 (t, 1H), 7.68 (d, 1H), 7.19 (d, 1H), 3.58-3.48 (m, 1H), 3.29-3.21 (m, 1H), 3.12-3.05 (m, 1H), 2.78-2.71 (m, 1H), 2.58-2.52 (m, 1H).

### Example 7A

### 5-Chloro-N-(2-oxiranylmethyl)-2-thiophenecarboxamide (racemic)

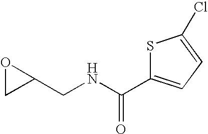

### Stage a): N-Allyl-5-chloro-2-thiophenecarboxamide

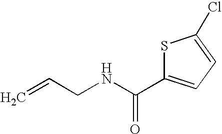

5.14 g (28 mmol) of 5-chlorothiophene-2-carbonyl chloride in 2 ml of absolute THF are added dropwise to an ice-cooled solution of 1.78 ml (24 mmol) of allylamine in 10 ml of absolute pyridine and 10 ml of absolute THF. The ice cooling is removed, and the mixture is stirred at room temperature for 2 h and then concentrated in vacuo. Water is added to the residue, and the resulting precipitate is filtered off, washed with water and dried under high vacuum.

Yield: 4.67 g (95% of theory)

LC-MS (method 2): Rₜ=2.98 min.

MS (ESIpos): m/z=202 [M+H]⁺.

### Stage b): 5-Chloro-N-(2-oxiranylmethyl)-2-thiophenecarboxamide

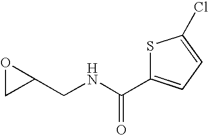

3.83 g of meta-chloroperbenzoic acid (approx. 60% pure) are added to an ice-cooled solution of 2.0 g (9.92 mmol) of N-allyl-5-chloro-2-thiophenecarboxamide in 10 ml of dichloromethane. The mixture is stirred while warming to room temperature overnight and then washed three times with 10% strength sodium bisulfate solution. The organic phase is washed twice with saturated sodium bicarbonate solution and with saturated sodium chloride solution, dried over magnesium sulfate and concentrated. The crude product is purified by chromatography on silica gel (mobile phase: cyclohexane/ethyl acetate 1:1).

Yield: 837 mg (39% of theory)

LC-MS (method 2): Rₜ=2.57 min.

MS (ESIpos): m/z=218 [M+H]⁺.

### Example 8A

### 2-[(2S)-2-Oxiranylmethyl]-1H-isoindole-1,3(2H)-dione

Obtainable by Mitsunobu reaction of (S)-(-)-2,3-epoxy-1-propanol with phthalimide, see A. Gutcait, K.-C. Wang, H.-W. Liu, L.-W. Chem, Tetrahedron Asym. 1996, 7, 1641-1648.

Exemplary Embodiments:

### [A] General method for preparing substituted N-(3-amino-2-hydroxypropyl)-5-chloro-2-thiophenecarboxamide derivatives starting from 5-chloro-N-(2-oxiranylmethyl)-2-thiophenecarboxamide

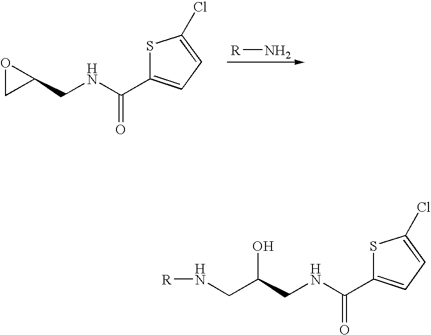

5-Chloro-N-[(2S)-(2-oxiranylmethyl)]-2-thiophenecarboxamide (1.0 eq.) is added in portions to a solution of primary amine or aniline derivative (1.0 to 2.0 eq.) in 1,4-dioxane, 1,4-dioxane/water mixtures, ethanol or ethanol/water mixtures (approx. 0.3 mol/l to 1.0 mol/l) at room temperature.

Alternative: 5-Chloro-N-[(2S)-(2-oxiranylmethyl)]-2-thiophenecarboxamide (1.2 eq.) and ytterbium(III) trifluoromethanesulfonate (0.1 eq.) are added to a solution of primary amine or aniline derivative (1.0 eq.) in THF (approx. 0.3 mol/l to 1.0 mol/l) at room temperature.

The respective reaction mixture is stirred at room temperature or at temperatures of up to 80° C. for 2 to 16 hours and then concentrated in vacuo. The product can be purified by chromatography on silica gel (mobile phase: cyclohexane/ethyl acetate mixtures, dichloromethane/methanol mixtures or dichloromethane/methanol/-triethylamine mixtures).

### Example 1

### 5-Chloro-N-({(5S)-2-oxido-3-[4-(3-oxo-4-morpholinyl)phenyl]-1,2,3-oxathia-zolidin-5-yl}methyl)-2-thiophenecarboxamide

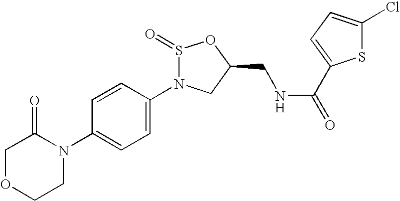

### Stage a): 5-Chloro-N-((2R)-2-hydroxy-3-{[4-(3-oxo-4-morpholinyl)-phenyl]amino}propyl)-2-thiophenecarboxamide

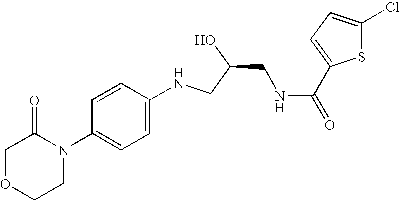

500 mg (2.6 mmol) of 4-(4-aminophenyl)morpholin-3-one are dissolved in 10 ml of THF and, at RT, 679.47 mg (3.1 mmol) of 5-chloro-N-[(2S)-2-oxiranylmethyl]-2-thiophenecarboxamide and 161.34 mg (0.3 mmol) of ytterbium(III) trifluoromethanesulfonate are added. The solution is stirred at 60° C. overnight. The precipitated white product is filtered off, washed with THF and dried under high vacuum. 574 mg (54% of theory) of the title compound are obtained. The filtrate is concentrated and the residue is purified by preparative HPLC (column: YMC gel ODS-AQ S-11 μm; mobile phase: water/acetonitrile, gradient 90:10→5:95). A further 402 mg (38% of theory) of the desired product are obtained in this way.

Yield: total 976 mg (92% of theory)

LC-MS (method 1): Rₜ=1.67 min.

MS (ESIpos): m/z=410 [M+H]⁺

¹H-NMR (200 MHz, DMSO-d₆): δ=8.62 (t, 1H), 7.68 (d, 1H), 7.18 (d, 1H), 7.02 (d, 2H), 6.59 (d, 2H), 5.66 (t, 1H), 5.09 (d, 1H), 4.13 (s, 2H), 3.96-3.88 (m, 2H), 3.86-3.74 (m, 1H), 3.64-3.55 (m, 1H), 3.30-2.90 (m, 2H).

### Stage b): 5-Chloro-N-({(5S)-2-oxido-3-[4-(3-oxo-4-morpholinyl)phenyl]-1,2,3-oxathiazolidin-5-yl}methyl)-2-thiophenecarboxamide

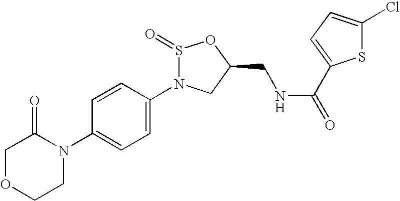

550 mg (1.3 mmol) of 5-chloro-N-((2R)-2-hydroxy-3-{[4-(3-oxo-4-morpholinyl)-phenyl]amino}propyl)-2-thiophenecarboxamide are dissolved in 40 ml of THF and, at −78° C. under argon, 2.34 ml (13.4 mmol) of N,N-diisopropylethylamine are added. 117.45 μl (1.6 mmol) of thionyl chloride, dissolved in 10 ml of THF, are added dropwise. The solution is stirred at RT overnight. The crude product after concentration of the solution is purified by preparative HPLC (column: YMC gel ODS-AQ S-11 μm; mobile phase: water/acetonitrile, gradient 90:10→5:95).

Yield: 392 mg (64% of theory)

LC-MS (method 1): Rₜ=1.88 min.

MS (ESIpos): m/z=456 [M+H]⁺

¹H-NMR (300 MHz, DMSO-d₆): δ=8.89 (t, 1H), 7.67 (d, 1H), 7.38 (d, 2H), 7.19 (d, 1H), 7.11 (d, 2H), 5.45-5.35 (m, 1H), 4.18 (s, 2H), 4.09-4.02 (m, 1H), 3.99-3.93 (m, 2H), 3.72-3.62 (m, 5H).

### Example 2

### 5-Chloro-N-({1-[4-(4-morpholinyl)phenyl]-5-oxo-3-pyrrolidinyl}methyl)-2-thio-phenecarboxamide

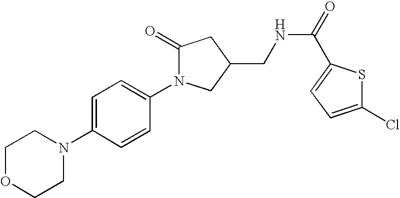

### Stage a): 1-[4-(4-Morpholinyl)phenyl]-5-oxo-3-pyrrolidinecarboxylic acid

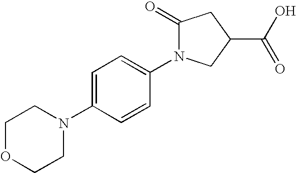

730 mg (5.61 mmol) of itaconic acid are dissolved in 6 ml of water, and 1000 mg (5.61 mmol) of 4-(4-morpholinyl)aniline are added to the solution. The reaction mixture is heated to reflux with stirring overnight. After cooling to room temperature, the reaction mixture is diluted with water and dichloromethane, the aqueous phase is extracted with dichloromethane, and the combined organic phases are dried over magnesium sulfate, filtered and concentrated. 1390 mg of the desired product are obtained and are directly reacted further.

### Stage b): Methyl 1-[4-(4-morpholinyl)phenyl]-5-oxo-3-pyrrolidinecarboxylate

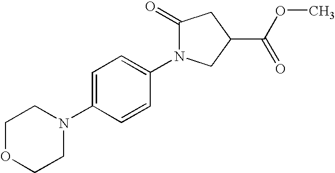

1390 mg (4.79 mmol) of 1-[⁴-(4-morpholinyl)phenyl]-5-oxo-3-pyrrolidinecarboxylic acid are dissolved in 40 ml of methanol and, at 0° C., 0.42 ml (5.57 mmol) of thionyl chloride is added. The reaction mixture is stirred at 0° C. for 1 h and at room temperature for 4 h and then concentrated. The residue is purified by chromatography on silica gel (mobile phase: ethanol/dichloromethane mixtures). 1158 mg of the desired product are obtained.

MS (ESIpos): m/z=305 [M+H]⁺

HPLC (method 3): Rₜ=2.95 min.

### Stage c): 4-(Hydroxymethyl)-1-[4-(4-morpholinyl)phenyl]-2-pyrrolidinone

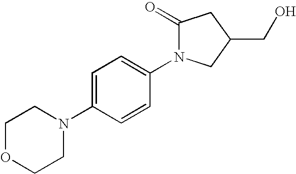

1105 mg (3.63 mmol) of methyl 1-[4-(4-morpholinyl)phenyl]-5-oxo-3-pyrrolidinecarboxylate are dissolved in 40 ml of methanol, and 412 mg (10.9 mmol) of sodium borohydride are added. The reaction mixture is heated to reflux with stirring for 6 h. After cooling to room temperature, the reaction mixture is acidified by cautious addition of 2N hydrochloric acid, and most of the methanol is removed under reduced pressure in a rotary evaporator. The residue is diluted with dichloromethane and made alkaline with 2N sodium hydroxide solution. The aqueous phase is extracted twice with dichloromethane, and the combined organic phases are dried over magnesium sulfate, filtered and concentrated. 998 mg of the desired product are obtained.

MS (ESIpos): m/z=277 [M+H]⁺

HPLC (method 3): Rₜ=2.23 min.

### Stage d): 2-({1-[4-(4-Morpholinyl)phenyl]-5-oxo-3-pyrrolidinyl}methyl)-1H-isoindole-1,3(2H)-dione

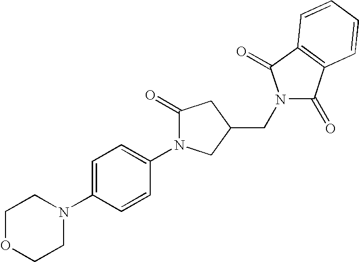

574 mg (3.9 mmol) of phthalimide and 1023 mg (3.9 mmol) of triphenylphosphine are dissolved in 20 ml of tetrahydrofuran, and a suspension of 980 mg (3.55 mmol) of 4-(hydroxymethyl)-1-[4-(4-morpholinyl)phenyl]-2-pyrrolidinone in a little tetrahydrofuran is added. The reaction mixture is cooled to 0° C., and 679 mg (3.9 mmol) of diethyl azodicarboxylate are added. The reaction mixture is stirred at 0° C. for 1 h and at room temperature for 4 h. It is then diluted with dichloromethane and washed with 1N sodium hydroxide solution. The organic phase is dried over magnesium sulfate, filtered and concentrated. The residue, which contains triphenylphosphine oxide in addition to the desired product, is employed without further purification in the next stage.

MS (ESIpos): m/z=406 [M+H]⁺

HPLC (method 3): Rₜ=3.53 min.

### Stage e): 4-(Aminomethyl)-1-[4-(4-morpholinyl)phenyl]-2-pyrrolidinone

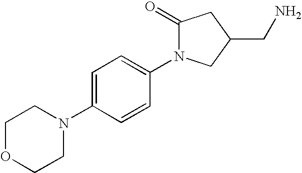

The crude product from the previous reaction [2-({1-[4-(4-morpholinyl)phenyl]-5-oxo-3-pyrrolidinyl}methyl)-1H-isoindole-1,3(2H)-dione, approx. 3.5 mmol] is dissolved in 20 ml of methanol, and 0.25 ml (5.25 mmol) of hydrazine monohydrate is added. The reaction mixture is heated to reflux with stirring overnight. After cooling to room temperature, the reaction mixture is diluted with dichloromethane and washed with 2N sodium hydroxide solution. The organic phase is dried over magnesium sulfate, filtered and concentrated. The crude product is employed without further purification in the next stage.

MS (ESIpos): m/z=276 [M+H]⁺.

### Stage f): 5-Chloro-N-({1-[4-(4-morpholinyl)phenyl]-5-oxo-3-pyrrolidinyl}-methyl)-2-thiophenecarboxamide

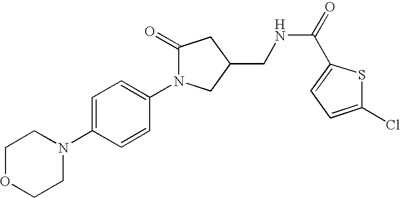

The crude product from the previous reaction [4-(aminomethyl)-1-[4-(4-morpholinyl)phenyl]-2-pyrrolidinone, approx. 0.8 mmol] is dissolved in 5 ml of tetrahydrofuran, and 0.2 ml (1.43 mmol) of triethylamine and 150 mg (0.83 mmol) of 5-chlorothiophene-2-carbonyl chloride are added. The reaction mixture is stirred at room temperature for 3 h, diluted with dichloromethane and washed with 2N sodium hydroxide solution. The organic phase is dried over magnesium sulfate, filtered and concentrated. The residue is purified by chromatography on silica gel (mobile phase: dichloromethane/ethanol mixtures). 170 mg of the desired product are obtained.

MS (ESIpos): m/z=420 [M+H]⁺

HPLC (method 3): Rₜ=3.49 min.

¹H-NMR (200 MHz, DMSO-d₆): δ=8.78 (t, 1H), 7.63 (d, 1H), 7.46 (d, 2H), 7.20 (d, 1H), 6.93 (d, 2H), 3.90 (dd, 1H), 3.72 (t, 4H), 3.58 (dd, 1H), 3.35-3.27 (m, 2H), 3.07 (t, 4H), 2.72-2.56 (m, 2H), 2.40-2.20 (m, 1H).

### Example 3

### 5-Chloro-N-({5-oxo-1-[4-(2-oxo-1-pyrrolidinyl)phenyl]-3-pyrrolidinyl}methyl)-2-thiophenecarboxamide

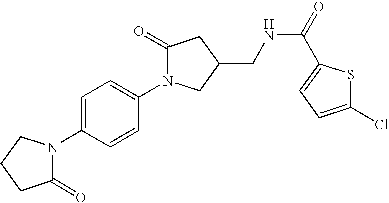

### Stage a): 5-Oxo-1-[4-(2-oxo-1-pyrrolidinyl)phenyl]-3-pyrrolidinecarboxylic acid

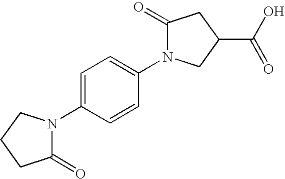

The title compound is obtained in analogy to example 2, stage a), by reacting 1-(4-aminophenyl)-2-pyrrolidinone with itaconic acid.

MS (ESIpos): m/z=289 [M+H]⁺

HPLC (method 3): Rₜ=2.53 min.

### Stage b): Methyl 5-oxo-1-[4-(2-oxo-1-pyrrolidinyl)phenyl]-3-pyrrolidine-carboxylate

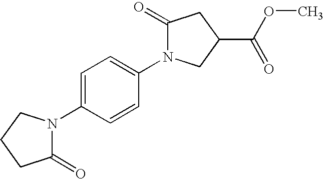

The title compound is obtained in analogy to example 2, stage b), by reacting 5-oxo-1-[4-(2-oxo-1-pyrrolidinyl)phenyl]-3-pyrrolidinecarboxylic acid with thionyl chloride in methanol.

MS (ESIpos): m/z=303 [M+H]⁺

HPLC (method 8): Rₜ=2.73 min.

### Stage c): 4-(Hydroxymethyl)-1-[4-(2-oxo-1-pyrrolidinyl)phenyl]-2-pyrrolidinone

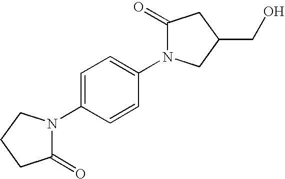

The title compound is obtained in analogy to example 2, stage c), by reacting methyl 5-oxo-1-[4-(2-oxo-1-pyrrolidinyl)phenyl]-3-pyrrolidinecarboxylate with sodium borohydride.

MS (ESIpos): m/z=275 [M+H]⁺

HPLC (method 3): Rₜ=2.39 min.

### Stage d): 2-({5-Oxo-1-[4-(2-oxo-1-pyrrolidinyl)phenyl]-3-pyrrolidinyl}methyl)-1H-isoindole-1,3(2H)-dione

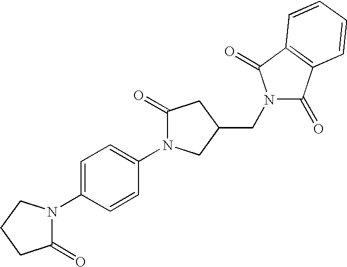

The title compound is obtained in analogy to example 2, stage d), by reacting 4-(hydroxymethyl)-1-[4-(2-oxo-1-pyrrolidinyl)phenyl]-2-pyrrolidinone with phthalimide.

MS (ESIpos): m/z=404 [M+H]⁺

HPLC (method 3): Rₜ=3.51 min.

### Stage e): 4-(Aminomethyl)-1-[4-(2-oxo-1-pyrrolidinyl)phenyl]-2-pyrrolidinone

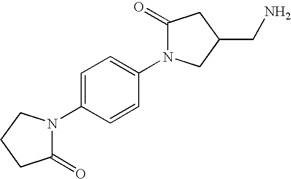

The title compound is obtained in analogy to example 2, stage e), by reacting 2-({5-oxo-1-[4-(2-oxo-1-pyrrolidinyl)phenyl]-3-pyrrolidinyl}methyl)-1H-isoindole-1,3(2H)-dione with hydrazine monohydrate.

### Stage f): 5-Chloro-N-({5-oxo-1-[4-(2-oxo-1-pyrrolidinyl)phenyl]-3-pyrroli-dinyl}methyl)-2-thiophenecarboxamide

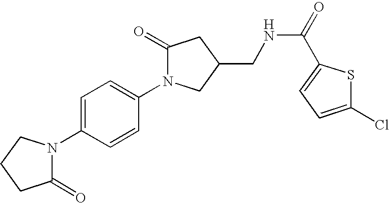

The title compound is obtained in analogy to example 1, stage f), by reacting 4-(aminomethyl)-1-[4-(2-oxo-1-pyrrolidinyl)phenyl]-2-pyrrolidinone with 5-chlorothiophene-2-carbonyl chloride.

MS (ESIpos): m/z=418 [M+H]⁺

HPLC (method 3): Rₜ=3.57 min.

¹H-NMR (200 MHz, DMSO-d₆): δ=8.79 (t, 1H), 7.70-7.58 (m, 5H), 7.20 (d, 1H), 3.95 (dd, 1H), 3.82 (t, 2H), 3.61 (dd, 1H), 3.38-3.25 (m, 2H), 2.75-2.49 (m, 2H), 2.50-2.28 (m, 3H), 2.15-1.97 (m, 2H).

### Example 4

### 5-Chloro-N-({5-oxo-4-[4-(2-oxo-1-pyrrolidinyl)phenyl]-2-morpholinyl}methyl)-2-thiophenecarboxamide

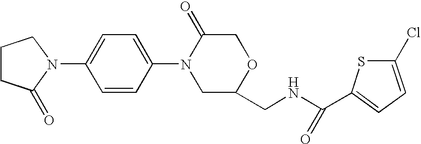

### Stage a): 5-Chloro-N-(2-hydroxy-3-{[4-(2-oxo-1-pyrrolidinyl)phenyl]amino}-propyl)-2-thiophenecarboxamide

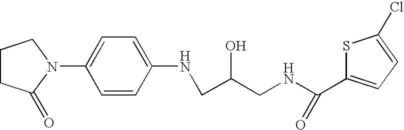

The title compound is prepared in accordance with general methods [A] by reacting 1-(4-aminophenyl)pyrrolidin-2-one with 5-chloro-N-(2-oxiranylmethyl)-2-thiophene-carboxamide in an ethanol/water mixture.

MS (DCI, NH₃): m/z=411 [M+NH₄]⁺

R_{f}=0.11 (ethyl acetate)

m.p.: 164° C.

¹H-NMR (200 MHz, DMSO-d₆): δ=8.59 (t, 1H), 7.68 (d, 1H), 7.28 (d, 2H), 7.17 (d, 1H), 6.58 (d, 2H), 5.40 (t, 1H), 5.02 (d, 1H), 3.87-3.76 (m, 1H), 3.72 (t, 2H), 3.41-3.18 (m, 2H), 3.16-3.03 (m, 1H), 3.01-2.88 (m, 1H), 2.40 (t, 2H), 2.09-1.97 (m, 2H).

### Stage b): 5-Chloro-N-({5-oxo-4-[4-(2-oxo-1-pyrrolidinyl)phenyl]-2-morpho-linyl}methyl)-2-thiophenecarboxamide

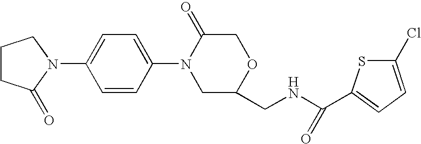

30 mg (1.13 mmol) of sodium hydride are added to a suspension of 400 mg (1.02 mmol) of 5-chloro-N-(2-hydroxy-3-{[4-(2-oxo-1-pyrrolidinyl)phenyl]amino}-propyl)-2-thiophenecarboxamide in 12 ml of THF under argon at room temperature and, after stirring for 30 minutes, 120 mg (1.02 mmol) of methyl chloroacetate are added dropwise over the course of 15 minutes. The reaction mixture is stirred at RT for 20 h, and the residue is filtered off and washed.

MS (ESIpos): mn/z=434 [M+H]⁺, 456 [M+Na]⁺

R_{f}=0.76 (ethanol)

m.p.: 201° C. (decomp.)

¹H-NMR (200 MHz, DMSO-d₆): δ=8.95 (t, 1H), 7.77 (d, 1H), 7.69 (d, 2H), 7.38 (d, 2H), 7.19 (d, 1H), 4.26 (s, 2H), 4.20-4.06 (m, 1H), 3.90-3.79 (dd, 2H), 3.78-3.58 (m, 4H), 3.53-3.41 (m, 2H), 2.13-1.98 (m, 2H).

### Example 5

### 5-Chloro-N-({(5S)-3-[4-(3-oxo-4-morpholinyl)phenyl]-2-thioxo-1,3-oxazolidin-5-yl}methyl)-2-thiophenecarboxamide

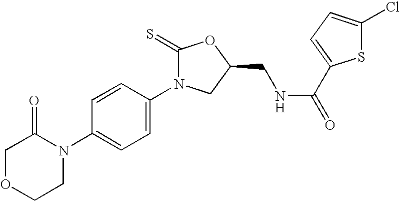

86 mg (0.2 mmol) of 5-chloro-N-((2R)-2-hydroxy-3-{[4-(3-oxo-4-morpholinyl)-phenyl]amino}propyl)-2-thiophenecarboxamide [example 1, stage a)] are dissolved in 5 ml of DMF, and 56.09 mg (0.3 mmol) of N,N′-thiocarbonyldiimidazole and 2.6 mg (0.02 mmol) of 4-N,N-dimethylaminopyridine are added. The solution is stirred at RT for 6 hours and then at 60° C. for 12 hours. The solution is concentrated and the residue is purified by preparative HPLC (column: YMC gel ODS-AQ S-11 μm; mobile phase: water/acetonitrile, gradient 90:10→5:95).

Yield: 26 mg (27% of theory)

LC-MS (method 5): Rₜ=2.07 min.

MS (ESIpos): m/z=452 [M+H]⁺

¹ H-NMR (300 MHz, DMSO-d₆):δ =8.99 (t, 1H), 7.70 (d, 1H), 7.63 (d, 2H), 7.47 (d, 2H), 7.20 (d, 1H), 5.12-5.02 (m, 1H), 4.42 (t, 1H), 4.41 (s, 2H), 4.16-4.07 (m, 1H), 4.01-3.95 (m, 2H), 3.78-3.72 (m, 2H), 3.65 (t, 2H).

### Example 6

### 5-Chloro-N-({(5S)-3-[4-(2-oxo-1-imidazolidinyl)phenyl]-2-thioxo-1,3-oxazolidin-5-yl}methyl)-2-thiophenecarboxamide

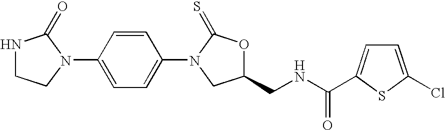

### Stage a): 5-Chloro-N-((2R)-2-hydroxy-3-{[4-(2-oxo-1-imidazolidinyl)phenyl]-amino}propyl)-2-thiophenecarboxamide

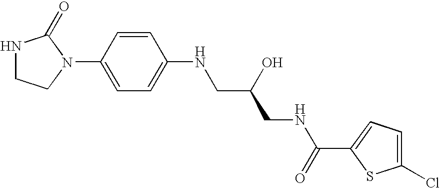

1.0 g (5.6 mmol) of 1-(4-aminophenyl)imidazolidin-2-one are dissolved in 10 ml of THF and, at RT, 1.47 g (6.8 mmol) of 5-chloro-N-[(2S)-2-oxiranylmethyl]-2-thiophenecarboxamide and 350 mg (0.6 mmol) of ytterbium(III) trifluoromethanesulfonate are added. The solution is stirred at 60° C. overnight. The solution is concentrated and the residue is purified by preparative HPLC (column: YMC gel ODS-AQ S-11 μm; mobile phase: water/acetonitrile, gradient 90:10→5:95).

Yield: 1.6 g (72% of theory)

LC-MS (method 4): Rₜ=1.39 min.

MS (ESIpos): m/z=395 [M+H]⁺.

### Stage b): 5-Chloro-N-({(5S)-3-[4-(2-oxo-1-imidazolidinyl)phenyl]-2-thioxo-1,3-oxazolidin-5-yl} methyl)-2-thiophenecarboxamide

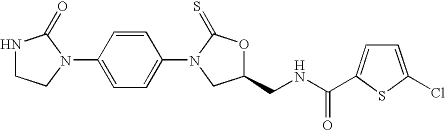

380 mg (0.2 mmol) of 5-chloro-N-((2R)-2-hydroxy-3-{[4-(2-oxo-1-imidazolidinyl)-phenyl]amino}propyl)-2-thiophenecarboxamide are dissolved in 10 ml of THF, and 343 mg (1.9 mmol) of N,N′-thiocarbonyldiimidazole and 11.76 mg (0.1 mmol) of 4-N,N-dimethylaminopyridine are added. The solution is stirred at RT for 6 hours and then at 60° C. for 12 hours. The precipitate is filtered off and washed with dichloromethane.

Yield: 94 mg (22% of theory)

LC-MS (method 6): R^{t}=2.07 min.

MS (ESIpos): m/z=437 [M+H]⁺.

¹H-NMR (200 MHz, DMSO-d₆): δ =9.02 (t, 1H), 7.71 (d, 1H), 7.63-7.56 (m, 2H), 7.52-7.44 (m, 2H), 7.22 (d, 1H), 7.02 (br. s, 1H), 5.12-5.00 (m, 1H), 4.36 (t, 1H), 4.11-4.00 (m, 1H), 3.91-3.80 (m, 2H), 3.65 (t, 2H), 3.35-3.30 (m, 2H).

### Example 7

### 5-Chloro-N-[((5S)-3-{4-[3-(2-hydroxyethyl)-2-oxotetrahydro-1(2H)-pyrimidinyl]phenyl}-2-thioxo-1,3-oxazolidin-5-yl)methyl]-2-thiophenecarboxamide

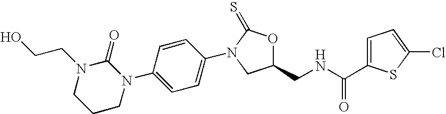

### Stage a): N-[(2R)-3-({4-[3-(2-{[tert-Butyl(diphenyl)silyl]oxy}ethyl)-2-oxotetra-hydro-1(2H)-pyrimidinyl]phenyl}amino)-2-hydroxypropyl]-5-chloro-2-thiophenecarboxamide

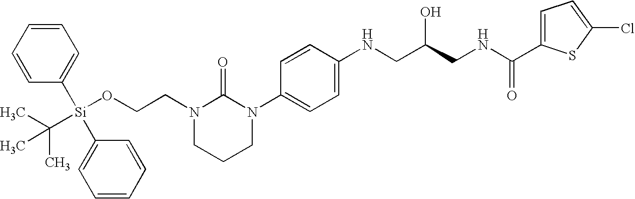

7.35 g (15.5 mmol) of 1-(4-aminophenyl)-3-(2-{[ter.-butyl(diphenyl)silyl]oxy}ethyl)tetrahydro-2(1H)-pyrimidinone are dissolved in 140 ml of THF and, at RT, 4.05 g (18.6 mmol) of 5-chloro-N-[(2S)-2-oxiranylmethyl]-2-thiophenecarboxamide and 962.40 mg (1.6 mmol) of ytterbium(III) trifluoromethanesulfonate are added. The solution is stirred at 60° C. overnight. The solution is concentrated and the residue is purified by chromatography on silica gel (mobile phase: dichloromethane/ethyl acetate 10:1→1:10).

Yield: 6.16 g (51% of theory)

LC-MS (method 1): Rₜ32 2.92 min.

MS (ESIpos): m/z=691 [M+H]⁺.

### Stage b): 5-Chloro-N-[((5S)-3-{4-[3-(2-hydroxyethyl)-2-oxotetrahydro-1(2H)-pyrimidinyl]phenyl}-2-thioxo-1,3-oxazolidin-5-yl) methyl]-2-thio-phenecarboxamide

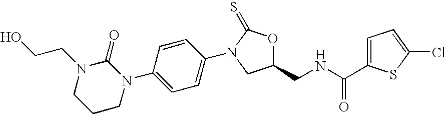

300 mg (0.4 mmol) of N-[(2R)-3-({4-[3-(2-{[tert-butyl(diphenyl)silyl]oxy}ethyl)-2-oxotetrahydro-1(2H)-pyrimidinyl]phenyl}amino)-2-hydroxypropyl]-5-chloro-2-thiophenecarboxamide are dissolved in 10 ml of THF, and 154.7 mg (0.9 mmol) of N,N′-thiocarbonyldiimidazole and 5.3 mg (0.04 mmol) of 4-N,N-dimethylaminopyridine are added. The solution is stirred at RT for 6 hours and then at 60° C. for 12 hours. The residue after concentration of the solution is dissolved in 10 ml of THF, and 868 μl (0.9 mmol) of tetra-n-butylammonium fluoride solution (1M in THF) are added. The solution is stirred at RT for 1 hour. The residue after concentration of the solution is dissolved in ethyl acetate/water (1:1). After separation, the organic phase is washed with saturated sodium chloride solution, dried and concentrated. The crude product is purified by preparative HPLC (column: YMC gel ODS-AQ S-11 μm; mobile phase: water/acetonitrile, gradient 90:10→5:95).

Yield: 63 mg (29% of theory)

LC-MS (method 5): Rₜ=2.00 min.

MS (ESIpos): m/z=496 [M+H]⁺

¹H-NMR (300 MHz, DMSO-d₆): δ =8.99 (t, 1H), 7.70 (d, 1H), 7.52-7.46 (m, 2H), 7.34-7.29 (m, 2H), 7.20 (d, 1H), 5.11-5.00 (m, 1H), 4.64 (t, 1H), 4.38 (t, 1H), 4.12-4.04 (m, 1H), 3.68-3.61 (m, 4H), 3.56-3.48 (m, 2H), 3.44 (t, 2H), 3.36-3.26 (m,2H), 2.06-1.96 (m, 2H).

B. Assessment of the Physiological Activity

The compounds of the formula (I) act in particular as selective inhibitors of coagulation factor Xa and do not inhibit, or also inhibit only at distinctly higher concentrations, other serine proteases such as thrombin, plasmin or trypsin.

Inhibitors of coagulation factor Xa are referred to as “selective” when their IC₅₀ values for factor Xa inhibition are 100-fold, preferably 500-fold, in particular 1000-fold, smaller than the IC₅₀ values for the inhibition of other serine proteases, in particular thrombin, plasmin and trypsin, reference being made concerning the test methods for the selectivity to the test methods of Examples B a.1) and a.2) described below.

The particularly advantageous biological properties of the compounds of the invention can be ascertained by the following methods.

a) Test Description (in vitro)

a.1) Measurement of Factor Xa Inhibition

The enzymatic activity of human factor Xa (FXa) was measured via the conversion of an FXa-specific chromogenic substrate. In this case, factor Xa eliminates p-nitroaniline from the chromogenic substrate. The determinations were carried out in microtiter plates as follows.

The test substances were dissolved in various concentrations in DMSO and incubated with human FXa (0.5 nmol/l dissolved in 50 mmol/l tris buffer [C,C,C-tris(hydroxymethyl)-aminomethane], 150 mmol/l NaCl, 0.1% BSA (bovine serum albumine), pH=8,3) at 25° C. for 10 minutes. Pure DMSO serves as control. The chromogenic substrate (150 μmol/l Pefachrome® FXa from Pentapharm) was then added. After incubation at 25° C. for 20 minutes, the extinction at 405 nm was determined. The extinctions of the test mixtures with test substance were compared with the control mixtures without test substance, and the IC₅₀ values were calculated therefrom.

a.2) Selectivity Determination

Selective FXa inhibition was demonstrated by investigating the inhibition by the test substances of other human serine proteases such as thrombin, trypsin, plasmin. The enzymatic activity of thrombin (75 mU/ml), trypsin (500 mU/ml) and plasmin (3.2 nmol/l) was determined by dissolving these enzymes in tris buffer (100 mmol/l, 20 mmol/l CaCl₂, pH=8.0) and incubating with test substance or solvent for 10 minutes. The enzymatic reaction was then started by adding the appropriate specific chromogenic substrates (Chromozym Thrombin® from Boehringer Mannheim, Chromozym Trypsin® from Boehringer Mannheim, Chromozym Plasmin® from Boehringer Mannheim), and the extinction was determined at 405 nm after 20 minutes. All determinations were carried out at 37° C. The extinctions of the test mixtures with test substance were compared with the control samples without test substance, and the IC₅₀ values were calculated therefrom.

a.3) Determination of the Anticoagulant Effect

The anticoagulant effect of the test substances was determined in vitro in human and rat plasma. For this purpose, human blood was collected in a 0.11 molar sodium citrate solution in the sodium citrate/blood mixing ratio of 1/9. The blood was thoroughly mixed after collection and centrifuged at about 4000 g for 15 minutes. The supernatant was removed by pipette. The prothrombin time (PT, synonym: Quick's test) was determined in the presence of varying concentrations of test substance or the appropriate solvent using a commercially available test kit (Neoplastin® from Boehringer Mannheim or Hemoliance® RecombiPlastin from Instrumentation Laboratory). The test compounds were incubated with the plasma at 37° C. for 3 minutes. Coagulation was then induced by adding thromboplastin, and the time of onset of coagulation was determined. The concentration of test substance which brings about a doubling of the prothrombin time was found.

b) Determination of the Antithrombotic Effect (in vivo)

b.1) Arteriovenous Shunt Model (rat)

Fasting male rats (strain: HSD CPB:WU) weighing 200-250 g were anesthetized with a Rompun/Ketavet solution (12 mg/kg/50 mg/kg). Thrombus formation was induced in an arteriovenous shunt by a method based on that described by Christopher N. Berry et al., Br. J. Pharmacol. (1994), 113, 1209-1214. For this purpose, the left jugular vein and the right carotid artery were exposed. An extracorporeal shunt was formed between the two vessels using a 10 cm-long polyethylene tube (PE 60). This polyethylene tube was secured in the middle by tying in a further 3 cm-long polyethylene tube (PE 160) which contained a roughened nylon thread forming a loop to produce a thrombogenic surface. The extracorporeal circulation was maintained for 15 minutes. The shunt was then removed and the nylon thread with the thrombus was immediately weighed. The blank weight of the nylon thread had been found before the start of the experiment. The test substances were administered either intravenously through the tail vein or orally by gavage to conscious animals before setting up the extracorporeal circulation.

C. Exemplary Embodiments of Pharmaceutical Compositions

The compounds of the invention can be converted into pharmaceutical preparations in the following ways:

Tablet:

Composition:

100 mg of the compound of example 1, 50 mg of lactose, 50 mg of microcristalline cellulose, 10 mg of polyvinylpyrrolidone (PVP), 10 mg of crosslinked Na carboxymethylcellulose and 2 mg of magnesium stearate.

Tablet weight 222 mg. Diameter 8 mm, radius of curvature 12 mm.

Production:

The mixture of active ingredient, lactose and cellulose is granulated with a 5% strength solution (m/m) of the PVP in water. The granules are dried and then mixed with the crosslinked Na carboxymethylcellulose and the magnesium stearate for 5 minutes. This mixture is compressed using a conventional tablet press.

Suspension which can be Administered Orally:

Composition:

1000 mg of the compound of example 1, 1000 mg of ethanol (96%), 400 mg of xanthan gum and 97.6 g of water.

10 g of oral suspension correspond to a single dose of 100 mg of the compound of the invention.

Production:

The xanthan gum is suspended in ethanol, and the active ingredient is added to the suspension. The water is added while stirring. The mixture is stirred for about 6 hours until the swelling of the xanthan gum is complete.

Solution which can be Administered Orally:

Composition

500 mg of the compound of example 1, 2.5 g of polysorbate and 97 g of polyethylene glycol 400.

20 g of oral solution corresponds to a single dose of 100 mg of the compound of the invention.

Production

The active ingredient is suspended by stirring in the mixture of polyethylene glycol and polysorbate. The stirring process is continued until the active ingredient has completely dissolved.

i.v. Solution:

The active ingredient is dissolved at a concentration below the saturation solubility in a physiologically tolerated solvent (e.g. isotonic saline, 5% glucose solution, 30% PEG 400 solution). The solution is sterilized by filtration and dispensed into sterile and pyrogen-free injection containers.

1. A compound of the formula (I) 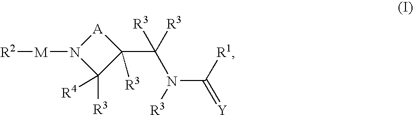
in which
A is a group 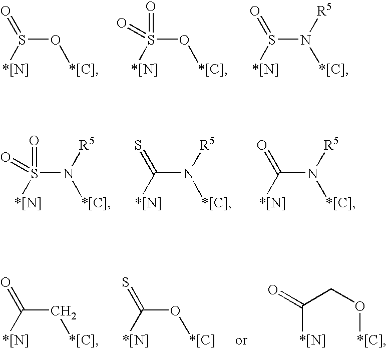
where
*[N] is the point of attachment to the nitrogen,
*[C] is the point of attachment to the carbon, and
R⁵ is hydrogen or alkyl,
M is an aryl, pyridyl, pyrimidyl pyridazinyl, thienyl, furyl or pyrrolyl radical which is unsubstituted or is substituted once or twice by radicals selected independently of one another from the group of halogen, trifluoromethyl, trifluoromethoxy, cyano, nitro, carbamoyl, hydroxy, amino, alkylcarbonyl, alkoxycarbonyl, optionally alkylamino-substituted alkylaminocarbonyl, alkylcarbonyloxy, alkyl, alkylamino and alkoxy,
where
alkyl, alkylamino and alkoxy in turn may be substituted by amino, hydroxy, alkylamino, alkoxy, heterocyclyl or heterocyclylcarbonyl,
R¹ is an aryl, heteroaryl or heterocyclyl radical which is unsubstituted or is substituted once, twice or three times by radicals selected independently of one another from the group of halogen, optionally amino-substituted alkyl, amino, alkylamino, hydroxy, alkoxy, alkoxycarbonyl, alkylcarbonyl, alkylcarbonyloxy, trifluoromethyl, trifluoromethoxy, trifluoromethylthio, nitro, oxo, carboxyl and cyano,
R² is an aryl, pyridyl, pyrimidyl or pyridazinyl radical,
which may be substituted by halogen, amino, alkylamino, alkylsulfonyl or alkyl-aminosulfonyl,
or
is an —N(R⁶)C(O)R⁷, —N(R⁸)C(O)NR⁹R¹⁰, —N(R¹¹)S(O)ₓR¹², 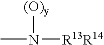
or
—C(O)NR¹⁵R⁶ radical,
where
R⁶, R⁸, R¹¹, R¹³ and R¹⁵ are independently of one another hydrogen, alkyl or cycloalkyl,
where
alkyl and cycloalkyl may in turn be substituted by amino, hydroxy, alkylamino or alkoxy,
R⁷, R⁹, R¹², R¹⁴ and R¹⁶ are independently of one another alkyl or cycloalkyl,
where
alkyl and cycloalkyl may in turn be substituted by amino, hydroxy, alkylamino or alkoxy,
or
R⁶ and R⁷ together with the N—C(O) group to which they are bonded form a 4- to 7-membered heterocycle which may also comprise one or two double bonds,
R⁸ and R⁹ together with the N—C(O)—N(R¹⁰) group to which they are bonded form a 5- to 7-membered heterocycle,
R¹⁰ is hydrogen, amino, hydroxy, alkylcarbonyl, alkylcarbonyloxy, alkoxycarbonyl, alkylaminocarbonyl, cycloalkyl, alkyl, alkylamino or alkoxy,
where
alkyl, alkylamino and alkoxy may in turn be substituted by amino, hydroxy, alkylamino, cycloalkylamino, alkoxy or heterocyclyl,
R¹¹ and R¹² together with the N—S(O)ₓ group to which they are bonded form a 4- to 7-membered heterocycle which may also comprise one or two double bonds,
R¹³ and R¹⁴ together with the nitrogen to which they are bonded form a 4- to 7-membered heterocycle,
R¹⁵ and R¹⁶ together with the nitrogen to which they are bonded form a 4- to 7-membered heterocycle,
where the heterocycle formed by R⁶ and R⁷; R⁸ and R⁹; R¹¹ and R¹²; R¹³ and R¹⁴ or by R¹⁵ and R¹⁶ comprises no, one, or two further heteroatoms from the series N, O and/or S and is unsubstituted or is substituted once, twice or three times by radicals independently of one another selected from the group of halogen, trifluoromethyl, cyano, nitro, amino, hydroxy, oxo, alkylcarbonyl, alkylcarbonyloxy, alkoxycarbonyl, alkylaminocarbonyl, alkyl, alkylamino and alkoxy,
where
alkyl, alkylamino and alkoxy may in turn be substituted by amino, hydroxy, alkylamino, alkoxy or heterocyclyl,
x is 1 or 2,
y is 0 or 1,
R³ is hydrogen or alkyl,
R⁴ is hydrogen, alkoxycarbonyl, alkylaminocarbonyl or alkyl,
where
alkyl in turn may be substituted by hydroxy, amino, alkoxy or alkylamino,
y is O or S
or a pharmaceutically acceptable salt thereof.

2. The compound as claimed in claim 1,
in which
A is a group 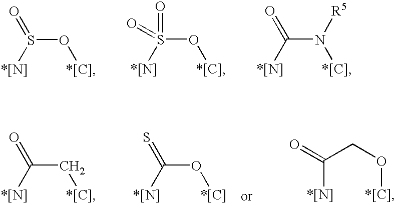
where
*[N] is the point of attachment to the nitrogen,
*[C] is the point of attachment to the carbon, and
R⁵ is hydrogen or methyl,
M is a phenyl or pyridyl radical which is optionally substituted once by fluorine, chlorine, trifluoromethyl, cyano, nitro, hydroxy, amino, acetyl, alkyl, alkylamino or alkoxy,
where
alkyl, alkylamino and alkoxy may in turn by substituted by amino, hydroxy, alkylamino, alkoxy or heterocyclyl,
R¹ is a phenyl, pyridyl, thienyl, furyl or pyrrolyl radical which is unsubstituted or is substituted once or twice by radicals selected independently of one another from the group of fluorine, chlorine, bromine, methyl, ethyl, aminomethyl, aminoethyl, amino, alkylamino, hydroxy, methoxy, acetyl, trifluoromethyl, trifluoromethoxy, trifluoromethylthio, nitro and cyano,
R² is a phenyl or pyridyl radical, which may be substituted by fluorine, chlorine, amino or alkylaminio,
or
is an —N(R⁶)C(O)R⁷, —N(R⁸)C(O)NR⁹R¹⁰, —N(R¹¹)S(O)ₓR¹², 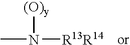
or —C(O)NR¹⁵R¹⁶ radical,
where
R⁶, R⁷, R⁸, R⁹, R¹¹, R¹², R¹³, R¹⁴, R¹⁵ and R¹⁶ are independently of one another methyl, ethyl, n-propyl, isopropyl, n-butyl, sec-butyl, isobutyl, tert-butyl, cyclopropyl or cyclopentyl,
each of which may in turn be substituted by amino, hydroxy, methoxy, ethoxy, methylamino, ethylamino, dimethylamino or diethylamino,
or
R⁶ and R⁷ together with the N—C(O) group to which they are bonded form a 5- or 6-membered heterocycle which may also comprise one or two double bonds,
R⁸ and R⁹ together with the N—C(O)—N(R¹⁰) group to which they are bonded form a 5- or 6-membered heterocycle,
R¹⁰ is hydrogen or alkyl,
where
alkyl may in turn be substituted by amino, hydroxy, alkylamino, cyclo-alkylamino, alkoxy or 5- or 6-membered heterocyclyl,
R¹¹ and R¹² together with the N—S(O), group to which they are bonded form a 5- or 6-membered heterocycle which may also comprise one or two double bonds,
R¹³ and R¹⁴ together with the nitrogen atom to which they are bonded form a 5- or 6-membered heterocycle,
R¹⁵ and R¹⁶ together with the nitrogen atom to which they are bonded form a 4- to 6-membered heterocycle,
where the heterocycle formed from R⁶ and R⁷; R⁸ and R⁹; R¹¹ and R¹²; R¹³ and R¹⁴ or from R¹⁵ and R¹⁶ optionally comprises a further heteroatom from the series N, O and/or S and is unsubstituted or is substituted once or twice by radicals selected independently of one another from the group of amino, hydroxy, oxo, acetyl, alkoxycarbonyl, alkylaminocarbonyl, alkyl, alkylamino and alkoxy,
where
alkyl, alkylamino and alkoxy may in turn be substituted by amino, hydroxy, alkylamino, alkoxy or 5- or 6-membered heterocyclyl,
x is 2,
y is 0,
R³ is hydrogen,
R⁴ is hydrogen or alkyl,
where
alkyl may in turn be substituted by hydroxy, amino, alkoxy or alkylamino,
Y is O,
or a pharmaceutically acceptable salt thereof.

3. The compound as claimed in claim 1 or 2,
in which
A is a group 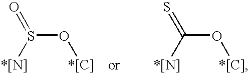
where
*[N] is the point of attachment to the nitrogen,
*[C] is the point of attachment to the carbon,
M is phenyl which is optionally substituted once by fluorine, chlorine, tri-fluoromethyl, cyano, amino, methyl, ethyl, methylamino or dimethylamino,
where
methyl and ethyl may in turn be substituted by amino, hydroxy, methylamino, dimethylamino, methoxy, morpholinyl, piperazinyl, piperidinyl or pyrrolidinyl,
R¹ is thienyl which is substituted once by chlorine, bromine or methyl,
R² is a radical 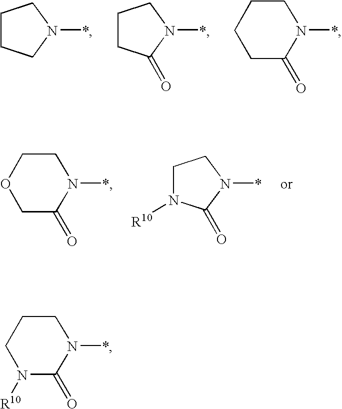
where
this radical is unsubstituted or is substituted once or twice by radicals selected independently of one another from the group of amino, hydroxy, methoxy, methylamino and dimethylamino,
* is the point of attachment to M,
and
R¹⁰ is hydrogen, methyl, ethyl or n-propyl,
where
ethyl and n-propyl may in turn be substituted by amino, hydroxy, methylamino, ethylamino, cyclopropylamino, isopropylamino, tert-butylamino, dimethylamino, diethylamino, methoxy, ethoxy, morpholinyl, piperazinyl, piperidinyl or pyrrolidinyl,
R³ is hydrogen,
R⁴ is hydrogen,
Y is O,
or a pharmaceutically acceptable salt thereof.

4. A process for preparing compounds as defined in claim 1, characterized in that either
(A) a compound of the formula (II) 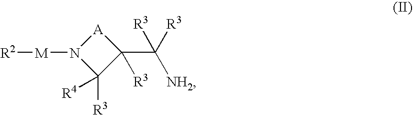
in which
A, M, R², R³ and R⁴ have the meanings indicated in claim 1,
is reacted with a compound of the formula (III) 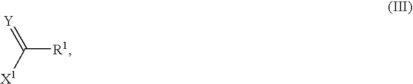
in which
R¹ and Y have the meanings indicated in claim 1, and
X¹ is chlorine or hydroxy,
or
(B) a compound of the formula (IV) 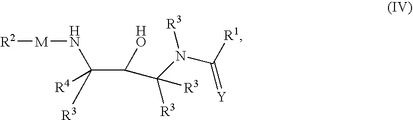
in which
M, R¹, R², R³, R⁴ and Y have the meanings indicated in claim 1, are reacted
(B1) with a compound of the formula (V) 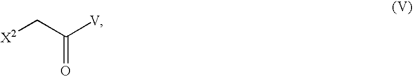
in which
V is alkoxy or chlorine, and
X² is a leaving group,
or
(B2) with thionyl chloride (SOCl₂)
or
(B3) with thionyl chloride (SOCl₂) and then with an oxidizing agent,
or
(B4) with N,N′-thiocarbonyldiimidazole
or
(C) a compound of the formula (VI) 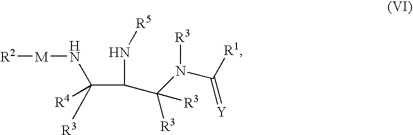
in which
M, R¹, R², R³, R⁴, R⁵ and Y have the meanings indicated in claim 1, is reacted
(C1) with a carbonic acid equivalent,
or
(C2) with thionyl chloride (SOCl₂)
or
(C3) with thionyl chloride (SOCl₂) and then with an oxidizing agent,
or
(C4) with N,N′-thiocarbonyldiimidazole,
and the resulting compound of the formula (I) is optionally reacted with an appropriate base or acid to give a pharmaceutically acceptable salt thereof.

5. (canceled)

6. A method for treating or preventing thromboembolic disorders, comprising administering to a patient a therapeutically effective amount of a compound of claim 1.

7. The method of claim 6, wherein said therapeutically effective amount has anticoagulant activity.

8. A method for preventing blood coagulation in vitro, characterized in that an amount having anticoagulant activity of a compound as defined in claim 1 is added.

9. (canceled)

10. A pharmaceutical composition comprising a compound as defined in claim 1 in combination with a pharmacologically acceptable excipient.

11. The pharmaceutical composition of claim 10, comprising a further active ingredient other than a compound as defined in claim 1.

12. The pharmaceutical composition of claim 10, wherein the further active ingredient is selected from the group consisting of
lipid-lowering agents;
coronary therapeutics/vasodilators;
plasminogen activators (thrombolyticslfibrinolytics) and compounds which increase thrombolysis/fibrinolysis,
substances having anticoagulant activity (anticoagulants);
platelet aggregation-inhibiting substances (platelet aggregation inhibitors); and
fibrinogen receptor antagonists (glycoprotein IIb/IIIa antagonists).

13. The pharmaceutical composition of claim 12, wherein the lipid-lowering agent is a HMG-CoA (3-hydroxy-3-methylglutaryl-coenzym A) reductase inhibitor.

14. The pharmaceutical composition of claim 12, wherein the coronary therapeutic/vasodilator is an ACE (angiotensin converting enzyme) inhibitor; AII (angiotensin II) receptor antagonist; β-adrenoceptor-antagonist; alpha-1-adrenoceptor antagonist; diuretic; calcium channel blocker; or a substance which brings about an increase in cyclic guanosine monophosphate (cGMP).

15. The pharmaceutical composition of claim 12, wherein the plasminogen activators (thrombolytics/fibrinolytics) and compounds which increase thrombolysis/fibrinolysis are inhibitors of plasminogen activator inhibitor (PAI inhibitors) or inhibitors of thrombin-activated fibrinolysis inhibitor (TAFI).
